(19) United States
(12) Patent Application Publication
(10) Pub. No.: US 2024/0277478 A1
(43) Pub. Date: Aug. 22, 2024

(54) CRANIOMAXILLOFACIAL IMPLANT AND METHOD OF DESIGNING THEREOF

(71) Applicant: METICULY COMPANY LIMITED, Bangkok (TH)

(72) Inventors: Kanin Sirichatchai, Bangkok (TH); Kantapat Phakdeewisetkul, Bangkok (TH); Tanya Charoensrisomboon, Bangkok (TH); Thanakorn Thiensri, Bangkok (TH)

(73) Assignee: METICULY COMPANY LIMITED, Bangkok (TH)

(21) Appl. No.: 18/568,491

(22) PCT Filed: Jun. 8, 2021
(86) PCT No.: PCT/TH2021/000031

§ 371 Date: Dec. 8, 2023

(51) Int. Cl. A61F 2/28; A61F 2/30

(52) U.S. Cl. CPC A61F 2/2875; A61F 2/30942; A61F 2002/2878; A61F 2002/2882

(57) ABSTRACT

A method of designing a craniomaxillofacial implant that is a rigid plate of substantially uniform thickness, including: positioning a surface element of the plate as a departure from a reference location within a base outline, the departure being represented by a fraction of the length of a normal path projected from the reference location to a predetermined soft tissue layer at a supported location within an anatomic region of interest, and the fraction being based upon a required patient-specific support at the supported location.

### FIELD OF THE INVENTION

The present invention relates to medical devices for bone surgery, particularly to human bone implants, and more particularly to the implants of craniomaxillofacial, i.e. the bones of human skull and face. The present invention further relates to methods of designing and manufacturing such devices, particularly to those which are computer-aided or computer-implemented.

### BACKGROUND OF THE INVENTION

Craniomaxillofacial implants find their applications in reconstruction and cosmetic surgeries. The two kinds of surgery have different objectives: a reconstruction surgery is performed to mend or replace a defective (i.e. unhealthy) part of the craniomaxillofacial bone which may have been caused by a trauma or disease; whereas a cosmetic surgery aims to improve the appearance of a patient's craniomaxillofacial features when there was no defective part originally. Regardless, being related to the patient's face—a body part of great significance to aesthetics and arguably to the quality of life—the patient's good postoperative appearance is important to both kinds of procedure. In fact, it is not uncommon that a reconstruction procedure is followed by at least a few rounds of cosmetic procedures, and that a patient is not considered fully healed until their facial appearance has been substantially restored. Because facial aesthetics are based upon the contralateral symmetry of the face, a slight damage to aesthetics is easily noticed and full restoration difficult to accomplish.

One challenge of craniomaxillofacial implantation, which conventionally requires many rounds of follow-up procedures, lies mainly with the relationship between the bone and nearby soft tissue layer(s). In the context of this field of technology, the particularly relevant soft tissue layers include fat, muscle, and/or skin. Said layer's atrophy and subsequent volume loss is particularly relevant to reconstruction surgeries. Being a gradual and unpredictable development, the final loss of volume tends to manifest over an uncertain period of time ranging from a course few weeks to a few years.

Another challenge which further restricts both kinds of craniomaxillofacial implantation is the trauma caused potentially by the procedure's invasiveness. The craniomaxillofacial region comprises only thin bones and soft tissue layers that cover the body's delicate and important organs, such as brain, sinus and eyeballs, along with the nerves that connect them. Ideally, a craniomaxillofacial implant should be small and thin; otherwise, the implantation would require a large incision which in turn cause a greater chance of atrophy and volume loss, which would later manifest and then affect more of the facial symmetry/aesthetics.

Said hindrance to a satisfactory postoperative facial aesthetics have been a long standing problem in the relevant field of technology.

Past attempts to address this problem include U.S. Pat. No. 9,216,084 B2 and U.S. Pat. No. 10,020,662 B2 of a common patent family (collectively, “Gordon et al.”) which teaches a patient-specific craniomaxillofacial implant, along with their designing method. Said implant is specifically directed to the reconstruction procedure (more specifically to the filling of the voids) of the cranium. Further, the implant comprises a base portion, having a first volume; and a preoperatively designed curved augmented portion to account for soft tissue loss overlying at least a portion of the void, having a second volume. The outer surface of said craniomaxillofacial implant is asymmetrical to contralateral bone of the skull. Said implant's main function being depended upon a partially augmented volume not only makes it bulky and invasive, but also makes its application limited to the cranium, a relatively large and thick portion of the craniomaxillofacial region which is the least affected by said invasiveness. Furthermore, Gordon et al.'s design of implant model is based upon the image of bone contralateral to the patient's cranial defect or pre-available data, which does not provide an accurate estimation of the loss of soft tissue.

Other notable past attempts include U.S. Pat. No. 9,895,211 B2 (“Yaremchuk”) and U.S. Pat. No. 10,792,141 B2 (“Brogan et al.”). Yaremchuk is directed to a craniofacial implant comprising a first body portion and a second body portion to be secured by a joining element, and a least one registration flange to prevent movement of the implant relative to the mandible in at least one direction when the implant is positioned adjacent the mandible. On the other hand, Brogan et al. is directed to a flexible polymer soft tissue temporal implant.

### SUMMARY OF THE INVENTION

An object of the present invention is to provide a novel craniomaxillofacial implant and its novel design method that is both capable of addressing the challenges common to the craniomaxillofacial region-loss of soft tissue layer and invasiveness- and applicable to multiple specific portions within the region.

Particular terms, including “postoperative image”, “template”, and “clinical outcomes”, are in accordance with the respective definitions provided in the Detailed Description section, further below.

In the first aspect, the present invention is embodied by a method of designing a craniomaxillofacial implant that is a rigid plate of substantially uniform thickness. Said method comprises positioning a surface element of said plate as a departure from a reference location within a base outline. Said departure is represented by a fraction of the length of a normal path projected from said reference location to a predetermined soft tissue layer at a supported location within an anatomic region of interest. And said fraction is based upon a required patient-specific support at said supported location.

The first aspect is related to a method necessary to realize a craniomaxillofacial implant in accordance with the first aspect. In particular, the first aspect pertains to the preoperative configuration required for the warpage of the plate of the implant in accordance with the second aspect (to be summarized below) in order to impart the implant with the utility to (i) be set onto and supported by at least a craniomaxillofacial bone and/or a soft tissue layer overlaying the craniomaxillofacial bone; and (ii) support the soft tissue layer in place of at least said craniomaxillofacial bone, thereby compensating or augmenting the elevation of the soft tissue layer.

Furthermore, while a design method in accordance with the prior art is based upon curves constructed from tangential lines which are determined from 2-dimension section planes of the images of the patient's craniomaxillofacial bone, a design method in accordance with the first aspect of the present invention is based upon the surface element which is positioned in terms of departure from the reference location. Such distinguishing feature of the first aspect allows a deeper customization to address a range of circumstantial patient needs, which may cover from that of local compensation to that of cosmetic purposes. Said feature also allows the topology/warpage of an embodiment to be configured in a sub-millimeter scale.

Preferably, the base outline is determined based on a postoperative craniomaxillofacial image and a preoperative craniomaxillofacial image.

Even more preferably, the base outline embodies a surface constraint which is determined based further on the curvature of soft tissue layer represented by said postoperative craniomaxillofacial image. Also even more preferably, the required patient-specific support at the supported location is further based on a volume difference between (i) the volume of the soft tissue layer as determined from the postoperative craniomaxillofacial image and (ii) the volume of the soft tissue layer as determined from the preoperative craniomaxillofacial image. The present inventors have found that implementing the foregoing references and bases, along with parameters determined therefrom, would significantly improve the outcomes of the method. The surface constraint that has been determined thus minimizes the area of based outline, and hence the invasiveness of the resulting implant. The required patient-specific support determined thus precisely represents the physical adjustments required for the intended surgical outcomes.

In an embodiment, the postoperative craniomaxillofacial image is obtained by registering a region of anatomical reference upon the preoperative craniomaxillofacial image. Said region of anatomical reference is defined correspondingly to a craniomaxillofacial region having the densest soft tissue layer that is also located closest to an operative craniomaxillofacial bone. The region of anatomical reference defined thus would take into account the nearby craniomaxillofacial region having the densest soft tissue layer. The deformation (atrophy, volume loss, etc.) of such region would most likely and significantly affect the precision of treatment.

Alternatively, the postoperative craniomaxillofacial image may be generated based on a mirrored contralateral craniomaxillofacial image; or, in the event that generating a mirrored image is not feasible (e.g. a case of frontal bone defect), said postoperative craniomaxillofacial image may be alternatively generated based on a craniomaxillofacial template that is searchable within a library containing a plurality of said templates. For an alternative involving the library, said library is preferably searchable by comparing anatomical landmarks of the preoperative craniomaxillofacial image with anatomical landmarks of the craniomaxillofacial templates contained within the library. Even more preferably, the library is configured to update the craniomaxillofacial template based on a feedback information comprising clinical outcomes data. For an embodiment whose library is configured to update the craniomaxillofacial template, it is most preferable that the update of the craniomaxillofacial template is carried out by an artificial intelligence.

It should be noted that an embodiment involving the library is particularly useful when the implant being designed is intended for a cosmetic surgery, or a reconstructive surgery wherein the contralateral craniomaxillofacial bone is damaged or otherwise unavailable. The library would provide a ready-to-use collection of references for the postoperative outcomes that cannot be found in the patient's own body (as in a mirrored craniomaxillofacial image).

Optionally, said fraction varies over a plurality of the reference locations within the base outline. Preferably, said fraction is further based upon a correction factor determined by an artificial intelligence trained of clinical outcomes data. The artificial intelligence trained thus further compensates the deformation of soft tissue layer that may develop over a longer period of time, if any, thereby reducing the aesthetical effects of such development and/or the risk of having to repeat the operation. Optionally, the length of said normal path varies over a plurality of the reference locations within the base outline. Also optionally, the required patient-specific support varies over a plurality of the reference locations within the anatomic region of interest.

Preferably, the required patient-specific support at the supported location is further based on the geometry and final position of the soft tissue layer. The present inventors have envisioned the importance of regarding the geometry and position as distinct parameters. This importance will be clarified hereinafter in an exemplary case related to an orbital implant.

An embodiment according to the first aspect may comprise further any one or any combination of the following steps: a step of configuring porosity of the plate; and a step of configuring mechanical reinforcement of the plate. Indeed, these steps are directed to the design and configuration of optional or preferable components of an implant in accordance with the second aspect, to be summarized below.

An embodiment according to the first aspect may be implemented to design a broad range of craniomaxillofacial implants. Exemplary cases of implant designed by such embodiments include: a cranial bone implant; a forehead implant; a maxilla implant; a zygomatic implant; an orbital implant; a nasal bone implant; a chin implant; and a mandible implant.

In the second aspect, the present invention is embodied by a craniomaxillofacial implant. Said implant is patient-specific and is formed into a plate that is rigid, warped, and of a substantially uniform thickness. Said warpage is configured preoperatively so as to adapt the implant to (i) be set onto and supported by at least a craniomaxillofacial bone and/or a soft tissue layer overlaying the craniomaxillofacial bone; and (ii) support the soft tissue layer in place of at least said craniomaxillofacial bone, thereby compensating or augmenting the elevation of the soft tissue layer.

According to the second aspect, the warped rigid plate is one of the implant's characteristic features which reduces the bulk as well as increases the adaptability for the purpose. Relying upon the warpage, not the augmented volume, keeps the plate/implant slim and of substantially uniform thickness throughout, thereby reducing the required size of incision and complications that may arise during the operation, e.g. pain, bleeding, and damage to nearby tissue. This second aspect finds its particular advantage when the patient has a thin or less flexible craniomaxillofacial skin, as with a patient who is elderly or afflicted with other health abnormalities. This group of patients would risk skin erosion caused by the invasiveness of an implant in accordance with the prior art, and thus would benefit from an implant in accordance with the second aspect.

The characteristic feature of warped rigid plate allows more customization. The present inventors also have envisioned that the topology/warpage of an embodiment may be configured in a sub-millimeter scale, which is enabled by the first aspect of the present invention, summarized above.

Said warpage may be configured preoperatively so as to also adapt the implant to support the soft tissue layer in place of at least said craniomaxillofacial bone, thereby compensating or augmenting the elevation of the soft tissue layer, said support being asymmetrical to that provided by a contralateral portion of the craniomaxillofacial bone. The warpage of the implant in accordance with this embodiment provides said contralateral asymmetry to account for the loss of soft tissue layer and/or to achieve the intended cosmetic outcomes, as the circumstances may require.

Preferably, said substantially uniform thickness is within a range of 0.2-0.8 mm. The implant is preferably made of a biocompatible material, particularly titanium or its compound/alloy, an alloplastic, or silicone; and also particularly a biocompatible ceramic, including hydroxyapatite.

The present inventors have envisioned that said soft tissue layer would be, in most cases, one or more of muscle, fat, and skin. This is because other species of soft tissue layer, which originally occupy less space and do not form a layer over the bone, would affect significantly less of the patient's facial aesthetics. Regardless, embodiments according to the present invention are applicable fully to other species of soft tissue layer.

Circumstantially, the surgery plan may require that the implant be set onto and supported by at least both the craniomaxillofacial bone and the soft tissue layer (i.e. both the craniomaxillofacial bone and soft tissue layer being disposed under the implant). An embodiment may be adapted in accordance with the present invention so as to meet said circumstantial requirement, as will be described later hereon.

Preferably, the plate is also porous, which further reduces the bulk and total weight. In some embodiments, the porosity may be even more preferably configured in a form of mesh. The reduced strength entailed by the porosity may be addressed by an embodiment further comprising a preoperatively configured mechanical reinforcement. Said mechanical reinforcement may take many forms, including those which fill the porosity locally, and those which thickens the plate locally. The location whereat the porosity is filled or the plate is thickened may be predetermined by a normal distribution to reinforce the plate's overall strength and/or by selectively locating the plate's high-load areas.

Preferably, the implant comprises further a means for fastening and/or increasing stability of the implant upon the craniomaxillofacial bone. An example of said means is a fixture protruding laterally from the plate's boundary, said fixture having an opening to receive a bone screw. Another notable example of said means is a fixture that is an opening for a bone screw, located within the plate's boundary. Any form of said fixture and its surrounding plate area may also be adapted to bend correspondingly with the topology of the patient's craniomaxillofacial bone and/or soft tissue layers so that the fixture may be mounted upon said bone and/or tissue with greater stability. Indeed, a main characteristic of embodiments in accordance with this second aspect, i.e. that the implant be formed into a plate that is rigid, warped, and of a substantially uniform thickness, allows such minute customization to the fixture so as to impart more utility than a simple screw hole.

Optionally, said warpage is configured preoperatively so as to adapt the implant to support the soft tissue layer by way of receiving said soft tissue layer in place of at least said craniomaxillofacial bone; or also optionally, said warpage is configured preoperatively so as to adapt the implant to support the soft tissue layer by way of lifting the soft tissue layer over at least said craniomaxillofacial bone. Said alternatives clarify that embodiments in accordance with the present invention may utilized in either a reconstruction surgery (i.e. wherein the implant substitutes the missing, deformed, or otherwise defective craniomaxillofacial bone) or a cosmetic surgery (i.e. wherein the implant modifies the support already provided by a healthy craniomaxillofacial bone).

An embodiment may be an implant for reconstructing a defective craniomaxillofacial bone and a defective soft tissue layer overlaying said defective craniomaxillofacial bone. Preferably, such embodiment has a warpage that is configured preoperatively so as to adapt the implant to (i) be set onto and supported by at least a non-defective part of the defective craniomaxillofacial bone and/or the defective part of said defective soft tissue layer; and (ii) support said defective soft tissue layer in place of the defective part of said defective craniomaxillofacial bone, and the defective part of said defective soft tissue layer. An exemplary case of said defective craniomaxillofacial bone is a cranium, the respective defective soft tissue layer comprising overlaying temporalis muscle and fat. Another exemplary case of said defective craniomaxillofacial bone is a maxilla or zygomatic, the respective defective soft tissue layer comprising overlaying zygomaticus major, zygomaticus minor, and fat. Yet another exemplary case of said defective craniomaxillofacial bone is an orbit, the respective defective soft tissue layer comprising overlaying orbicularis oculi, inferior rectus muscle, inferior oblique, eyeball and fat.

Further, an embodiment may be an implant for a cosmetic surgery. Preferably, such embodiment has a warpage is configured preoperatively so as to adapt the implant to (i) be set onto and supported by at least the craniomaxillofacial bone and/or the soft tissue layer; and (ii) support the soft tissue layer in place of at least said craniomaxillofacial bone, thereby altering the apparent contours of the craniomaxillofacial bone. An exemplary case of said cosmetic surgery is operated upon a nasal bone or a forehead. Another exemplary case of said cosmetic surgery is operated upon a maxilla or a zygoma. Yet another exemplary case of cosmetic surgery is operated upon a mandible.

### BRIEF DESCRIPTION OF DRAWINGS

The principle of the present invention and its advantages will become apparent in the following description, taking into consideration the accompanying drawings in which:

FIG. 1 shows a schematic flowchart representing a method of designing a craniomaxillofacial implant in accordance with a preferred embodiment.

FIG. 2 shows a schematic flowchart representing the step of generating a postoperative craniomaxillofacial image in accordance with a preferred embodiment.

FIG. 3A shows a schematic flowchart representing the step of obtaining a template of craniomaxillofacial image in accordance with the first alternative of a preferred embodiment.

FIG. 3B shows a schematic flowchart representing the step of obtaining a template of craniomaxillofacial image in accordance with the second alternative of a preferred embodiment.

FIG. 4 shows a front view of the postoperative craniomaxillofacial image obtained by the step of registering the region of anatomical reference upon the aligned preoperative craniomaxillofacial image in accordance with a preferred embodiment (not to scale).

FIG. 5A shows a perspective view of the preoperative craniomaxillofacial image that has been segmented in accordance with a preferred embodiment (not to scale).

FIG. 5B shows a perspective view of the postoperative craniomaxillofacial image that has been segmented in accordance with a preferred embodiment (not to scale).

FIG. 6 shows a schematic flowchart representing the step of determining a base outline in accordance with a preferred embodiment.

FIG. 7A shows a boundary and a surface constraint determined as part of the step of determining the base outline in accordance with a preferred embodiment (not to scale).

FIG. 7B shows an anatomic region of interest determined as part of the step of determining the base outline in accordance with a preferred embodiment (not to scale).

FIG. 8 shows a schematic flowchart representing the step of determining a normal path from the base outline to a supported location in accordance with a preferred embodiment.

FIG. 9A shows an assignment of the base outline in a scenario that the soft tissue layer to be supported includes muscle, fat and skin, in accordance with a preferred embodiment (not to scale).

FIG. 9B shows an assignment of the base outline in a scenario that the soft tissue layer to be supported includes only fat and skin, in accordance with a preferred embodiment (not to scale).

FIG. 10A shows an assignment of the supported location in a scenario where (1) the soft tissue layer to be supported includes muscle, fat and skin; (2) an edge of the implant being a closed supportive edge, in accordance with a preferred embodiment (not to scale).

FIG. 10B shows an assignment of the supported location in a scenario where (1) the soft tissue layer to be supported includes only fat and skin; (2) an edge of the implant being a closed supportive edge, in accordance with a preferred embodiment (not to scale).

FIG. 10C shows an assignment of the supported location in a scenario where (1) the soft tissue layer to be supported includes muscle, fat and skin; (2) an edge of the implant being an open supportive edge, in accordance with a preferred embodiment (not to scale).

FIG. 10D shows an assignment of the supported location in a scenario where (1) the soft tissue layer to be supported includes only fat and skin; (2) an edge of the implant being an open supportive edge, in accordance with a preferred embodiment (not to scale).

FIG. 11 shows a schematic image representing normal paths from the base outline to a supported location, determined in accordance with a preferred embodiment (not to scale).

FIG. 12 shows a schematic flowchart representing the step of determining a volume difference of the soft tissue layer to be supported in accordance with a preferred embodiment.

FIG. 13A shows a perspective front view representing the difference between the preoperative and postoperative volumes of the soft tissue layer of the same type, calculated in accordance with a preferred embodiment (not to scale).

FIG. 13B shows a sectioned rear view representing the difference between the preoperative and postoperative volumes of the soft tissue layer of the same type, calculated in accordance with a preferred embodiment (not to scale).

FIG. 14 shows a schematic flowchart representing the step of determining a departure from a reference location along the normal path in accordance with a preferred embodiment.

FIG. 15A shows departures from the reference locations along the normal paths with respect to the first axis of the surface constraint curvatures, determined when the relevant bone is a cranium, in accordance with a preferred embodiment (not to scale).

FIG. 15B shows departures from the reference locations along the normal paths with respect to the second axis of the surface constraint curvatures, determined when the relevant bone is a cranium, in accordance with a preferred embodiment (not to scale).

FIG. 15C shows departures from the reference location along the normal path with respect to the boundary of the implant being designed, determined when the relevant bone is a cranium, in accordance with a preferred embodiment (not to scale).

FIG. 16 shows a schematic flowchart representing the step of positioning a surface element in accordance with a preferred embodiment.

FIG. 17 shows an image of a cranial implant, the surface of which being formed by positioning the surface elements in accordance with a preferred embodiment (not to scale).

FIG. 18A shows an exemplary embodiment where the reference location coincides with a craniomaxillofacial bone (not to scale).

FIG. 18B shows an exemplary embodiment where the reference location coincides with a soft tissue layer (not to scale).

FIG. 19 shows a schematic flowchart representing the step of configuring porosity and mechanical reinforcement in accordance with a preferred embodiment.

FIG. 20A shows a schematic image of an implant, the porosity and mechanical reinforcement of which being configured in accordance with the first alternative of a preferred embodiment (not to scale).

FIG. 20B shows a schematic image of an implant, the porosity and mechanical reinforcement of which being configured in accordance with the second alternative of a preferred embodiment (not to scale).

FIG. 21 shows variations of the modes of mechanical reinforcement of in accordance with a preferred embodiment (not to scale).

FIG. 22 shows an assignment of the base outline in a scenario that the soft tissue layer to be supported includes only fat and muscle, in accordance with a preferred embodiment (not to scale).

FIG. 23A shows departures from the reference locations along the normal paths with respect to the first axis of the surface constraint curvatures, determined when the relevant bone is an orbit, in accordance with a preferred embodiment (not to scale).

FIG. 23B shows departures from the reference locations along the normal paths with respect to the second axis of the surface constraint curvatures, determined when the relevant bone is an orbit, in accordance with a preferred embodiment (not to scale).

FIG. 23C shows departures from the reference location along the normal path with respect to the boundary of the implant being designed, determined when the relevant bone is an orbit, in accordance with a preferred embodiment (not to scale).

FIG. 24 shows an image of an orbital implant, the surface of which being formed by positioning the surface elements in accordance with a preferred embodiment (not to scale).

FIG. 25A shows departures from the reference locations along the normal paths with respect to the first axis of the surface constraint curvatures, determined when the relevant bone includes a maxilla and a zygoma, in accordance with a preferred embodiment (not to scale). 5 FIG. 25B shows departures from the reference locations along the normal paths with respect to the second axis of the surface constraint curvatures, determined when the relevant bone includes a maxilla and a zygoma, in accordance with a preferred embodiment (not to scale).

FIG. 25C shows departures from the reference location along the normal path with respect to the boundary of the implant being designed, determined when the relevant bone includes a maxilla and a zygoma, in accordance with a preferred embodiment (not to scale).

FIG. 26 shows an image of a maxilla and zygomatic implant, the surface of which being formed by positioning the surface elements in accordance with a preferred embodiment (not to scale).

FIG. 27A shows departures from the reference locations along the normal paths with respect to an axis of the surface constraint curvatures, determined when the relevant is a nasal bone, in accordance with a preferred embodiment (not to scale).

FIG. 27B shows departures from the reference locations along the normal paths with respect to the boundary of the implant being designed, determined when the relevant is a nasal bone, in accordance with a preferred embodiment (not to scale).

FIG. 28 shows an image of a nasal bone implant, the surface of which being formed by positioning the surface elements in accordance with a preferred embodiment (not to scale).

FIG. 29A schematically shows the application of a cranial implant in accordance with a preferred embodiment (not to scale).

FIG. 29B schematically shows the warpage of a cranial implant in accordance with a preferred embodiment (not to scale).

FIG. 30A shows a front perspective view of a cranial implant in accordance with a preferred embodiment (not to scale).

FIG. 30B shows a rear perspective view of a cranial implant in accordance with a preferred embodiment (not to scale).

FIG. 31A schematically shows the application of an orbital implant in accordance with a preferred embodiment (not to scale).

FIG. 31B schematically shows the warpage of an orbital implant in accordance with a preferred embodiment (not to scale).

FIG. 32A shows a front perspective view of an orbital implant in accordance with a preferred embodiment (not to scale).

FIG. 32B shows a rear perspective view of an orbital implant in accordance with a preferred embodiment (not to scale).

FIG. 33A schematically shows the application of a maxilla-zygomatic implant in accordance with a preferred embodiment (not to scale).

FIG. 33B schematically shows the warpage of a maxilla-zygomatic implant in accordance with a preferred embodiment (not to scale).

FIG. 34A shows a front perspective view of a maxilla-zygomatic implant in accordance with a preferred embodiment (not to scale).

FIG. 34B shows a rear perspective view of a maxilla-zygomatic implant in accordance with a preferred embodiment (not to scale).

FIG. 35A schematically shows the application of a nasal bone implant in accordance with a preferred embodiment (not to scale).

FIG. 35B schematically shows the warpage of a nasal bone implant in accordance with a preferred embodiment (not to scale).

FIG. 36A shows a front perspective view of a nasal bone implant in accordance with a preferred embodiment (not to scale).

FIG. 36B shows a rear perspective view of a nasal bone implant in accordance with a preferred embodiment (not to scale).

FIG. 37 schematically shows the application of a mandibular implant in accordance with a preferred embodiment (not to scale).

### DETAILED DESCRIPTION OF EMBODIMENTS OF THE INVENTION

It is to be understood that the following detailed description will be directed to embodiments, provided as examples for illustrating the concept of the present invention only. The present invention is in fact not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section.

Unless defined otherwise, all technical and scientific terms used herein have the same meaning as commonly understood by one of ordinary skills in the art to which this invention belongs.

It must be noted that as used herein and in the appended claims, the singular forms “a”, “an”, and “the” include plural referents unless the context clearly dictates otherwise.

The term “about” when used before a numerical designation, e.g., dimensions, time, amount, and such other, including a range, indicates approximations which may vary by (+) or (−) 10%, 5% or 1%, or any sub-range or sub-value there between.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a device or method consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

“Patient-specific configuration” refers to one or more configurations applicable to a specified feature of embodiment that is specific to an individual patient's needs. Particularly, the term is intended to cover configurations directly apparent from an image of the bone and/or tissue as well as those obtainable by processing any information originating from the patient, so long that such configuration may be applied to part of an embodiment. More particularly, notable instances of patient-specific configuration in the present application includes the dimensions, topology and warpage of a craniomaxillofacial implant in accordance with an embodiment.

“Soft tissue” or “soft tissue layer” refers to a bodily tissue that has an adaptable cellular structure and contour, particularly upon a pressure or flexure. Generally, a soft tissue is inclusive of a blood vessel, fat, tendon, ligament, muscle, nerve, and skin; among which, muscle, fat, and skin will be mostly mentioned in the following parts of detailed description. But the other species of soft tissue are by no means excluded from the scope of this invention unless expressly indicated otherwise.

“Postoperative image”, as in “postoperative craniomaxillofacial image”, and the like, refers to a medical image of the specified body part that is a visualization of such body part after the operation of the relevant surgical procedure. A postoperative image according to this meaning is determined before the operation by way of image processing (mirroring, registration, rendering etc.) and/or from a library of images, as opposed to an image that is taken after the actual operation.

“Template”, when used in connection with a bone or other body parts, refers to a pre-visualized image of the corresponding bone or body part that represents the ideal results of the operation.

“Clinical outcomes” refers to medical and health conditions that are consequential to a treatment, i.e. a surgical operation, over any period of time. The term shall include any observable condition which may be a quantitative or qualitative condition. A qualitative condition is inclusive of but not limited to a medical image. In a case where the clinical outcomes is a medical image, said image is that taken from the patient after the actual operation, thereby being distinguished from the above-defined “postoperative image”.

### A Method of Designing a Craniomaxillofacial Implant

The first aspect of the present invention is directed to a novel method necessary to enable the novel craniomaxillofacial implants in accordance with the second aspect. It should be noted that the order of the steps and their subcomponents as shown in the following drawings and mentioned in the following description may be modified by a relevant skilled person upon full knowledge of the Detailed Description and drawings.

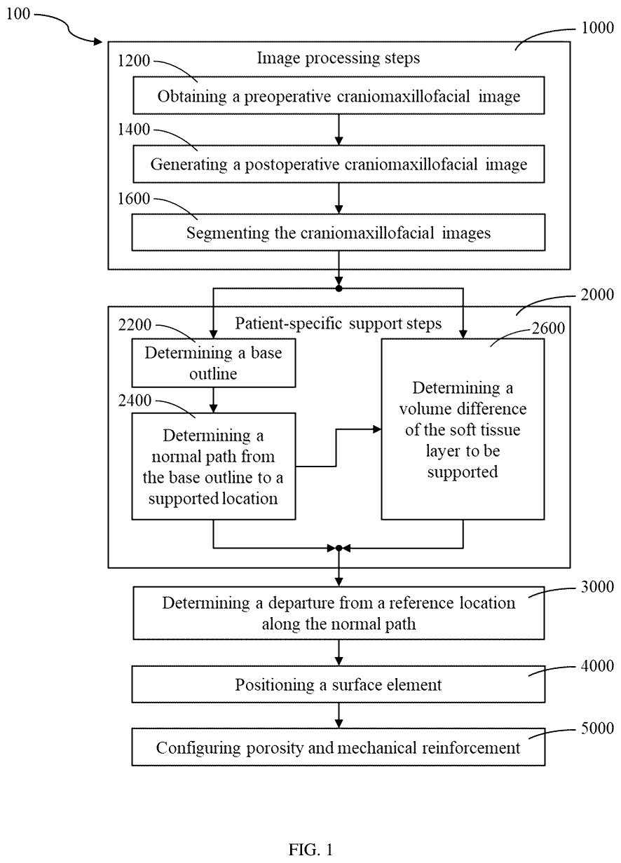

FIG. 1 shows a schematic flowchart representing a method of designing a craniomaxillofacial implant in accordance with a preferred embodiment. The craniomaxillofacial implant being designed by this embodiment is a rigid plate of substantially uniform thickness. In this embodiment, the method 100 begins with image processing steps 1000, which further comprises subcomponents of obtaining a preoperative craniomaxillofacial image 1200, generating a postoperative craniomaxillofacial image 1400, and then segmenting the craniomaxillofacial images 1600. This embodiment of the method 100 also comprises patient-specific support steps 2000, which further comprises subcomponents of determining a base outline 2200, determining a normal path from the base outline to a supported location 2400, and determining a volume difference of the soft tissue layer to be supported 2600. In this embodiment, the step of determining a base outline 2200 processes the information obtained by segmenting the craniomaxillofacial images 1600; the step of determining a normal path from the base outline to a supported location 2400 processes the information obtained by determining a base outline 2200; and the step of determining a volume difference of the soft tissue layer to be supported 2600 processes the information obtained by segmenting the craniomaxillofacial images 1600 and by determining a normal path from the base outline to a supported location 2400. According to this embodiment, information obtained from all the subcomponents 2200, 2400, 2600 of the patient-specific support steps 2000 is further processed by a step of determining a departure from a reference location along the normal path 3000. The preferred method 100 then proceeds to the steps of positioning a surface element 4000, and further to a step of configuring porosity and mechanical reinforcement 5000.

The subcomponent step of obtaining a preoperative craniomaxillofacial image 1200 as shown in FIG. 1 may be carried out by preoperatively performing a suitable medical imaging technique known in the relevant art upon the patient's relevant craniomaxillofacial bone. Said known technique includes X-ray fluoroscopy, computed tomography (CT), and magnetic resonance imaging (MRI).

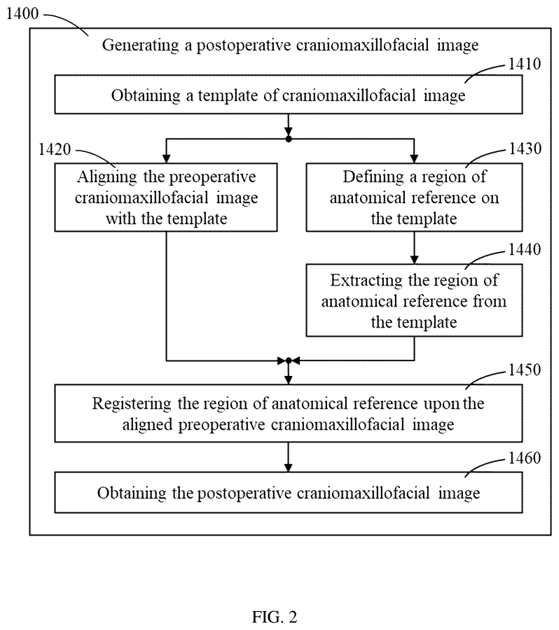

FIG. 2 shows a schematic flowchart representing the step of generating a postoperative craniomaxillofacial image in accordance with a preferred embodiment. Here, the step of generating a postoperative craniomaxillofacial image 1400, previously shown in FIG. 1, further comprises the following subcomponents: obtaining a template of craniomaxillofacial image 1410, the template obtained thus being subsequently processed by steps of aligning the preoperative craniomaxillofacial image with the template 1420 and defining a region of anatomical reference on the template 1430. Said region of anatomical reference is then extracted from the template by the respective step 1440. Information obtained from the foregoing subcomponents 1410, 1420, 1430, 1440 is further processed in a step of registering the region of anatomical reference upon the aligned preoperative craniomaxillofacial image 1450. Then, the postoperative craniomaxillofacial image is obtained by a respective step 1460.

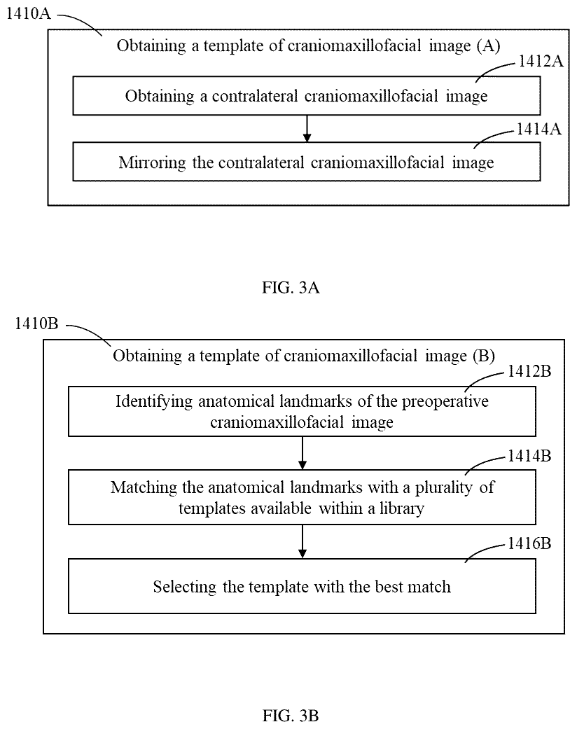

The step of obtaining a template of craniomaxillofacial image 1410, as shown in FIG. 2, may be carried out by preferred alternatives as shown in FIGS. 3A and 3B.

FIG. 3A shows a schematic flowchart representing such first alternative in accordance with a preferred embodiment. In this first alternative, the template of craniomaxillofacial image is obtained by mirroring the contralateral craniomaxillofacial image, i.e. the side that is not subject to the operation. Accordingly, the first alternative of obtaining a template of craniomaxillofacial image 1410A comprises subcomponent steps of obtaining a contralateral craniomaxillofacial image 1412A, and then mirroring the contralateral craniomaxillofacial image 1414A. The step 1410A may be carried out by preoperatively performing a suitable medical imaging technique known in the relevant art upon the patient's contralateral craniomaxillofacial bone. Said known technique includes X-ray fluoroscopy, computed tomography (CT), and magnetic resonance imaging (MRI). Also, the step 1410A may be carried out simultaneously with the above-mentioned step of obtaining a preoperative craniomaxillofacial image 1200 and with the same medical imaging technique. This first alternative of obtaining a template of craniomaxillofacial image 1410A finds its particular use when the purpose of the relevant operation is to reconstruct a defective craniomaxillofacial bone, provided that the patient's contralateral bone is unaffected by the defect.

FIG. 3B shows a schematic flowchart representing such second alternative in accordance with a preferred embodiment. In this second alternative, a plurality of templates are contained within a library. Said plurality of templates are not based upon the patient's own craniomaxillofacial image, but rather based upon the past cases of operation and/or are artificially generated. In the preferred embodiment, the template is selected from a plurality thereof based upon the matching between the anatomical of the patient's craniomaxillofacial bone and the templates' corresponding anatomical landmarks. Notable examples of said anatomical landmarks include: zygomatic arch, mandible condyle, nasal bone, and bone suture. The patient's anatomical landmarks may be identified from the preoperative craniomaxillofacial image obtained from the abovementioned respective step 1200. Preferably, the library is a digital library having the plurality of templates pre-loaded thereupon, said preferred library also being searchable by comparing anatomical landmarks of the preoperative craniomaxillofacial image with anatomical landmarks of the craniomaxillofacial templates contained within the library. Accordingly, the second alternative of obtaining a template of craniomaxillofacial image 1410B comprises subcomponent steps of identifying anatomical landmarks of the preoperative craniomaxillofacial image 1412B, matching the anatomical landmarks with a plurality of templates available within the library 1414B, and selecting the template with the best match 1416B. More preferably, the library is configured to update the craniomaxillofacial templates based on feedback information comprising clinical outcomes data. Said update of the craniomaxillofacial template is most preferably carried out by an artificial intelligence, which may involve any one or combination of machine learning-based algorithm, statistical shape modeling (SSM), multi-objective shape optimization, and topology optimization. This second alternative of obtaining a template of craniomaxillofacial image 1410B finds its particular use when the implant being designed is intended for a cosmetic surgery, or a reconstructive surgery wherein the contralateral craniomaxillofacial bone is damaged or otherwise unavailable. The library would provide a ready-to-use collection of references for the postoperative outcomes that cannot be found in the patient's own body (as in a mirrored craniomaxillofacial image).

The step of aligning the preoperative craniomaxillofacial image with the template 1420 as described earlier in connection with FIG. 2 is related to an image data alignment technique, i.e. incorporating two image data into a single frame of reference, which is already known in the relevant art. Said known technique includes voxel-based registration, surface-based registration, and iterative closest point algorithm (ICP).

The region of anatomical reference as defined on the template in accordance with the respective step 1430 and then extracted from the template in accordance with the respective step 1440, described earlier in connection with FIG. 2, corresponds to a craniomaxillofacial region having the densest soft tissue layer that is also located closest to the operative craniomaxillofacial bone. In exemplary embodiments, if the operative bone is a cranium, then the region of anatomical reference corresponds to a zygoma and/or a temporal bone; if the operative bone is an orbit, then the region of anatomical reference corresponds to a sphenoid bone and/or a frontal process of the maxilla; if the operative bone is a maxilla-zygomatic bone (i.e. a midface bone), then the region of anatomical reference corresponds to a maxilla bone and/or a frontal bone; and if the operative bone is a nasal bone, then the region of anatomical reference corresponds to the nasal bone, a maxilla, and/or a frontal bone. After the extraction, said region of anatomical reference is then registered in accordance with the respective step 1450 upon the preoperative craniomaxillofacial image that has been previously aligned with the template in accordance with the respective step 1420. Then, the image registered thus becomes the postoperative craniomaxillofacial image, obtained in accordance with the respective step 1460. An example of a postoperative craniomaxillofacial image obtained thus is shown in FIG. 4.

FIG. 4 shows a front view of the postoperative craniomaxillofacial image obtained by the step of registering the region of anatomical reference upon the aligned preoperative craniomaxillofacial image in accordance with a preferred embodiment. Here, the postoperative craniomaxillofacial image 1452 is a product from registering the regions of anatomical reference 1456 (shaded) upon the preoperative craniomaxillofacial image 1454 (unshaded) that has been aligned with the template. In this exemplary embodiment, the operation is related to a reconstruction of the patient's damaged right cranium, and thus the region of anatomical reference is determined to be the right zygomatic bone 1458. Both said right cranium and said right zygomatic bone 1458 are covered by the regions of anatomical reference 1456 (shaded).

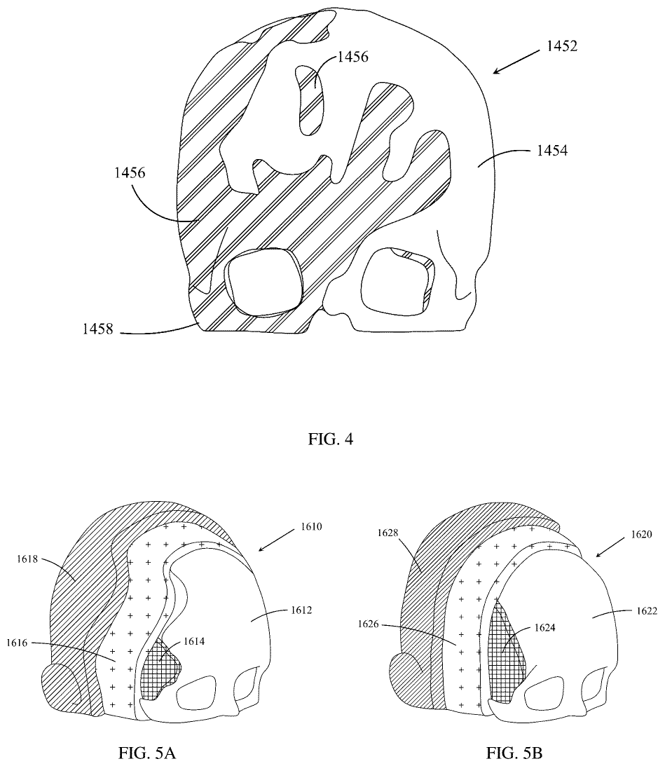

The step of segmenting the craniomaxillofacial images 1600, as previously mentioned in connection with FIG. 1, is related to an image processing technique employed to recognize and distinguish layers of soft tissue layers from a medical image. Known examples of such image processing techniques include: region-growing, adaptive thresholding, global thresholding, level-set method, and K-means clustering. In a preferred embodiment, said segmentation is performed upon both the preoperative craniomaxillofacial image, as obtained by the respective step 1200; and upon the postoperative craniomaxillofacial image, as generated by the respective step 1400. Examples of preoperative and postoperative craniomaxillofacial image segmented thus are shown in FIGS. 5A and 5B, respectively.

FIG. 5A shows a perspective view of the preoperative craniomaxillofacial image that has been segmented in accordance with a preferred embodiment; on the other hand, FIG. 5B shows a perspective view of the postoperative craniomaxillofacial image that has been segmented in accordance with a preferred embodiment. In both of these exemplary embodiments, the operation is related to a reconstruction of the patient's damaged right cranium.

Particularly, FIG. 5A shows a preoperative craniomaxillofacial image 1610 which, after the segmentation, reveals, from the innermost to the outermost layer, the craniomaxillofacial bone 1612, the muscle layer 1614 which in this embodiment is a temporalis muscle, the fat layer 1616, and the skin layer 1618. Likewise, FIG. 5B shows a postoperative craniomaxillofacial image 1620 which, after the segmentation, reveals, from the innermost to the outermost layer, the craniomaxillofacial bone 1622, the muscle layer 1624, the fat layer 1626, and the skin layer 1628.

In an alternative embodiment, the operation is related to the patient's orbit and the muscle layer is an eyeball, orbicularis oculi, inferior rectus muscle, and/or inferior oblique; in yet another alternative embodiment, the operation is related to the patient's maxilla-zygomatic bone and the muscle layer is a zygomaticus major and/or a zygomaticus minor.

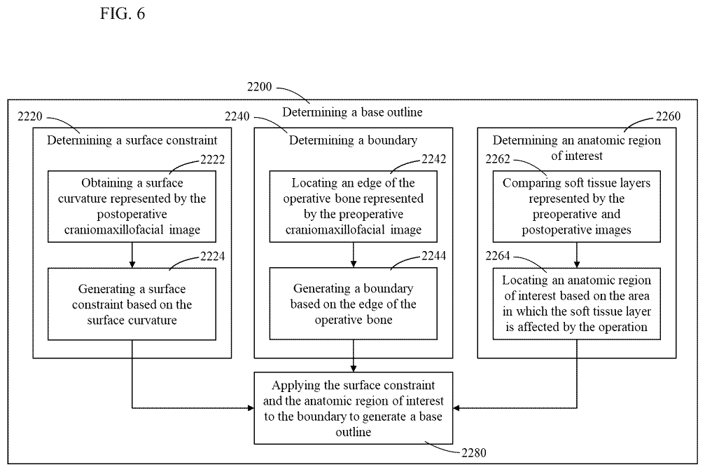

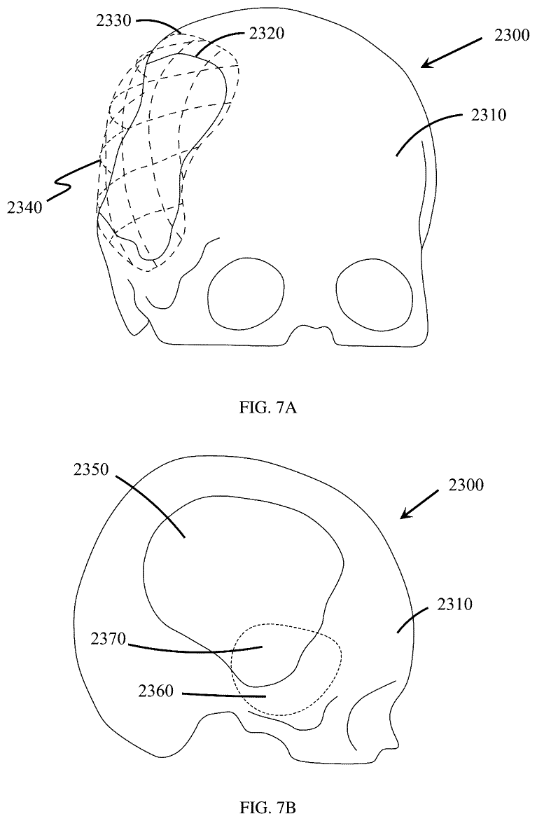

Next, FIG. 6 shows a schematic flowchart representing the step of determining a base outline in accordance with a preferred embodiment. Here, the step of determining a base outline 2200, previously shown in FIG. 1, further comprises the following subcomponents: determining a surface constraint 2220, determining a boundary 2240, and determining an anatomic region of interest 2260; the surface constraint and anatomic region of interest determined by the respective subcomponent steps 2220, 2260 are subsequently applied to the boundary determined by the respective subcomponent step 2240 to generate a base outline in the respective subcomponent step 2280. Furthermore, the subcomponent of determining a surface constraint 2220 is carried out by obtaining a surface curvature represented by the postoperative craniomaxillofacial image 2222, and then generating a surface constraint based on the surface curvature 2224; the subcomponent of determining a boundary 2240 is carried out by locating an edge of the operative bone represented by the preoperative craniomaxillofacial image 2242, and then generating a boundary based on the edge of the operative bone 2244; finally, the subcomponent of determining an anatomic region of interest 2260 is carried out by comparing soft tissue layers represented by the preoperative and postoperative images 2262, and then locating an anatomic region of interest based on the area in which the soft tissue layer is affected by the operation 2264. The detail and products of the foregoing subcomponent steps and their related actions are depicted by FIGS. 7A and 7B.

FIG. 7A shows a boundary and a surface constraint determined as part of the step of determining the base outline in accordance with a preferred embodiment. Here, the preoperative craniomaxillofacial image 2300 represents a patient's craniomaxillofacial bone 2310 having a damaged right cranium, which is the operative bone in this embodiment. The damaged right cranium is defined by an edge of the operative bone 2320. FIG. 7A shows further that a boundary 2330 is determined to enclose the edge of the operative bone 2320 and that a surface constraint 2340 is determined to represent the surface curvature within the area of the postoperative craniomaxillofacial image that corresponds to the boundary 2330. The foregoing boundary 2330 and the surface constraint 2340 amount to two out of three elements required for the preferred base outline determined in accordance with the respective subcomponent step 2200. Next, FIG. 7B will show the remaining elements: the anatomic region of interest.

FIG. 7B shows an anatomic region of interest determined as part of the step of determining the base outline in accordance with a preferred embodiment. Here, the preoperative craniomaxillofacial image 2300 represents a view from the right side of the same patient's craniomaxillofacial bone 2310 as shown earlier in FIG. 7A. In FIG. 7B, the edge of the operative bone 2320 defines the operative bone 2350 which is the damaged right cranium. FIG. 7B further shows an area defined by broken lines, which represents an interested portion of the soft tissue layer 2360 that is affected by the operation as determined by comparing the soft tissue layers represented by the preoperative and postoperative images. The area where the operative bone 2350 intersects the interested portion of the soft tissue layer 2360 is determined to be an anatomic region of interest 2370. Said anatomic region of interest 2370 is subsequently projected to the boundary 2330 (shown in FIG. 7A) as modified by the surface constraint 2340 (shown in FIG. 7A) to define the area within the implant where a reference location is assigned, and from which the surface element departs, as will be more apparent in the later part of the detailed description.

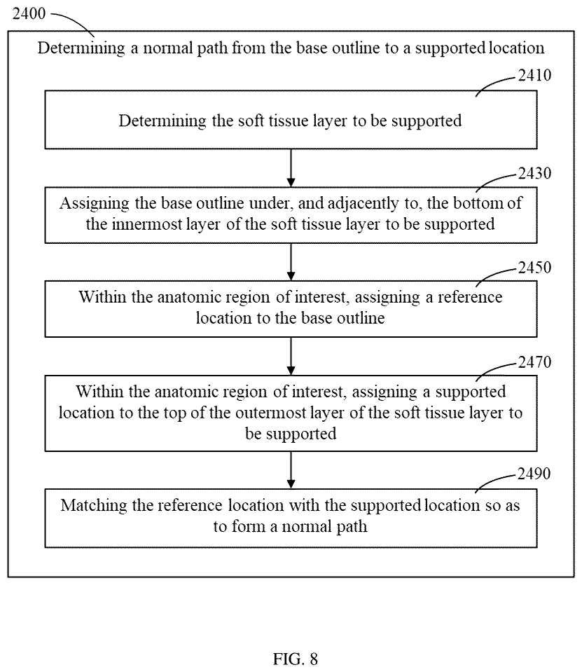

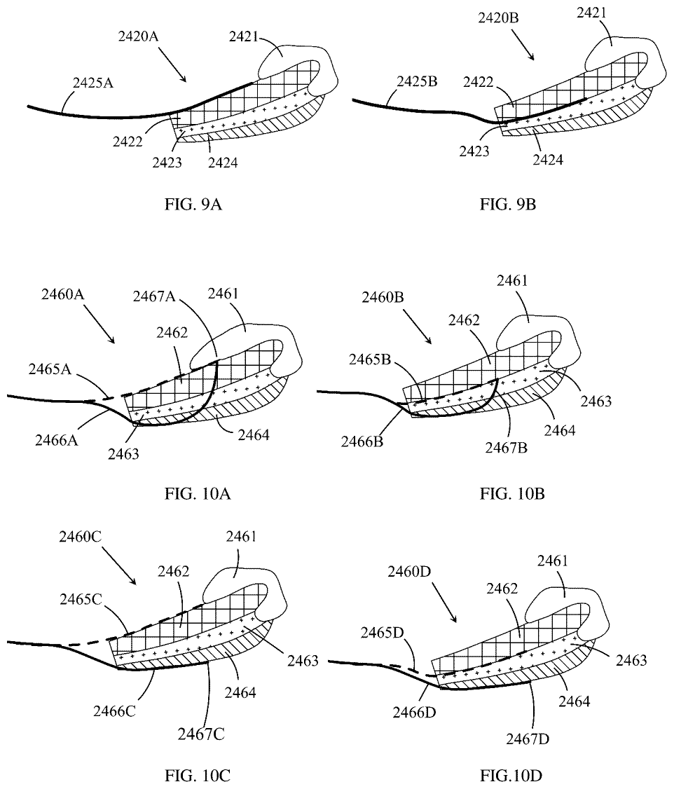

FIG. 8 shows a schematic flowchart representing the step of determining a normal path from the base outline to a supported location in accordance with a preferred embodiment. Here, the step of determining a normal path from the base outline to a supported location 2400, previously shown in FIG. 1, further comprises the following subcomponents: determining the soft tissue layer to be supported 2410; assigning the base outline under, an adjacently to, the bottom of the innermost layer of the soft tissue layer to be supported 2430; then, within the anatomic region of interest, assigning a reference location to the base outline 2450; afterwards, within the anatomic region of interest, assigning a supported location to the top of the outermost layer of the soft tissue layer to be supported 2470; and finally, matching the reference location with the supported location so as to form a normal path 2490. These subcomponent steps provide the implant being designed with a wide range of customization regarding the manner of support for the soft tissue layer, which will be shown in the following FIGS. 9A, 9B, 10A, 10B, 10C, 10D, and 11, all of which pertain to exemplary embodiments where the patient's operative bone is the damaged right cranium.

FIGS. 9A and 9B exemplifies the possible results in accordance with the subcomponent steps of determining the soft tissue layer to be supported 2410; and assigning the base outline under, and adjacently to, the bottom of the innermost layer of the soft tissue layer to be supported 2430.

FIG. 9A shows a schematic cross-section postoperative image of the right cranium 2420A comprising, from the innermost to the outermost layer: a bone layer 2421, a muscle layer 2422 which in this embodiment is a temporalis muscle, a fat layer 2423, and a skin layer 2424. Here, the soft tissue layer to be supported is determined by the respective subcomponent step to include all the muscle 2422, fat 2423 and skin 2424 layers; thus, the base outline 2425A is assigned by the respective subcomponent step to be under, and adjacently to, the bottom of the innermost layer of the soft tissue to be supported, which in this case is the bottom of the muscle layer 2422. It is also to be appreciated that if the implant being designed were to depart from the base outline 2425A, such departure would affect the support imparted to all the muscle 2422, fat 2423 and skin 2424 layers.

On the other hand, FIG. 9B shows a schematic cross-section postoperative image of the right cranium 2420B comprising, from the innermost to the outermost layer: a bone layer 2421, a muscle layer 2422 which in this embodiment is a temporalis muscle, a fat layer 2423, and a skin layer 2424. Alternatively, the soft tissue layer to be supported here is determined by the respective subcomponent step to include only the fat 2423 and skin 2424 layers; thus, the base outline 2425B is assigned by the respective subcomponent step to be under, and adjacently to, the bottom of the innermost layer of the soft tissue to be supported, which in this case is the bottom of the fat layer 2423. In such case, the muscle layer 2422 is left unsupported and one may also appreciate that if the implant being designed were to depart from the base outline 2425B, such departure would affect the support imparted to the fat 2423 and skin 2424 layers only.

FIGS. 10A, 10B, 10C, and 10D exemplifies the possible results in accordance with the subcomponent steps carried out within the anatomic region of interest: assigning a reference location to the base outline 2450; and assigning a supported location to the top of the outermost layer of the soft tissue layer to be supported 2470. It should be noted that said anatomic region of interest has been determined according to the descriptions related to FIGS. 6, 7A, and 7B.

FIG. 10A shows a continuation from FIG. 9A in the first alternative. Here, a schematic cross-section postoperative image of the right cranium 2460A comprises, from the innermost to the outermost layer: a bone layer 2461, a muscle layer 2462 which in this embodiment is a temporalis muscle, a fat layer 2463, and a skin layer 2464. A base outline has been assigned to the bottom of the muscle layer 2462, to which location a reference location 2465A is assigned by the respective subcomponent step. Further, a supported location 2466A is assigned by the respective subcomponent step to the top of the outermost layer of the soft tissue layer to be supported, which in this case is the top of the skin layer 2464. The supported location 2466A indicates the geometry and final position of the soft tissue layer that is expected of the operation, hence the maximum support that is possible for the case being, but not necessarily the outline that the implant being designed actually follows. The anatomic region of interest covers where the lines representing the reference location 2465A and the supported location 2466A diverge. In this alternative, the supported location 2466A is predetermined to converge with the reference location 2465A, forming a closed supportive edge 2467A that is supported by the bone 2461.

FIG. 10B shows a continuation from FIG. 9B in the first alternative. Here, a schematic cross-section postoperative image of the right cranium 2460B comprises, from the innermost to the outermost layer: a bone layer 2461, a muscle layer 2462 which in this embodiment is a temporalis muscle, a fat layer 2463, and a skin layer 2464. A base outline has been assigned to the bottom of the fat layer 2463, to which location a reference location 2465B is assigned by the respective subcomponent step. Further, a supported location 2466B is assigned by the respective subcomponent step to the top of the outermost layer of the soft tissue layer to be supported, which in this case is the top of the skin layer 2464. The supported location 2466B indicates the geometry and final position of the soft tissue layer that is expected of the operation, hence the maximum support that is possible for the case being, but not necessarily the outline that the implant being designed actually follows. The anatomic region of interest covers where the lines representing the reference location 2465B and the supported location 2466B diverge. In this alternative, the supported location 2466B is predetermined to converge with the reference location 2465B, forming a closed supportive edge 2467B that is supported by the muscle layer 2462.

FIG. 10C shows a continuation from FIG. 9A in the second alternative. Here, a schematic cross-section postoperative image of the right cranium 2460C comprises, from the innermost to the outermost layer: a bone layer 2461, a muscle layer 2462 which in this embodiment is a temporalis muscle, a fat layer 2463, and a skin layer 2464. A base outline has been assigned to the bottom of the muscle layer 2462, to which location a reference location 2465C is assigned by the respective subcomponent step. Further, a supported location 2466C is assigned by the respective subcomponent step to the top of the outermost layer of the soft tissue layer to be supported, which in this case is the top of the skin layer 2464. The supported location 2466C the geometry and final position of the soft tissue layer that is expected of the operation, hence the maximum support that is possible for the case being, but not necessarily the outline that the implant being designed actually follows. The anatomic region of interest covers where the lines representing the reference location 2465C and the supported location 2466C diverge. In this alternative, the supported location 2466C is predetermined to remain diverged from the reference location 2465C, forming an open supportive edge 2467C which is unsupported by any of the bone 2461, the muscle layer 2462, the fat layer 2463, and the skin layer 2464.

FIG. 10D shows a continuation from FIG. 9B in the second alternative. Here, a schematic cross-section postoperative image of the right cranium 2460D comprises, from the innermost to the outermost layer: a bone layer 2461, a muscle layer 2462 which in this embodiment is a temporalis muscle, a fat layer 2463, and a skin layer 2464. A base outline has been assigned to the bottom of the fat layer 2463, to which location a reference location 2465D is assigned by the respective subcomponent step. Further, a supported location 2466D is assigned by the respective subcomponent step to the top of the outermost layer of the soft tissue layer to be supported, which in this case is the top of the skin layer 2464. The supported location 2466D indicates the geometry and final position of the soft tissue layer that is expected of the operation, hence the maximum support that is possible for the case being, but not necessarily the outline that the implant being designed actually follows. The anatomic region of interest covers where the lines representing the reference location 2465D and the supported location 2466D diverge. In this alternative, the supported location 2466D is predetermined to remain diverged from the reference location 2465D, forming an open supportive edge 2467D which is unsupported by any of the bone 2461, the muscle layer 2462, the fat layer 2463, and the skin layer 2464.

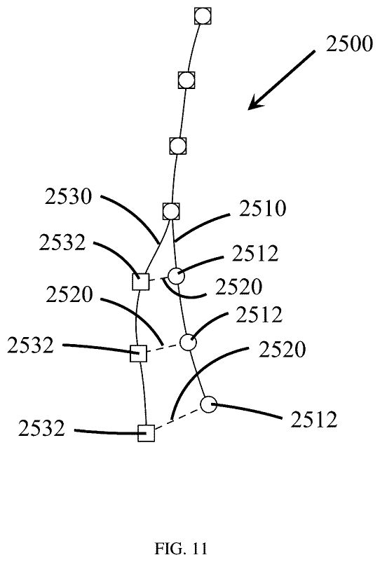

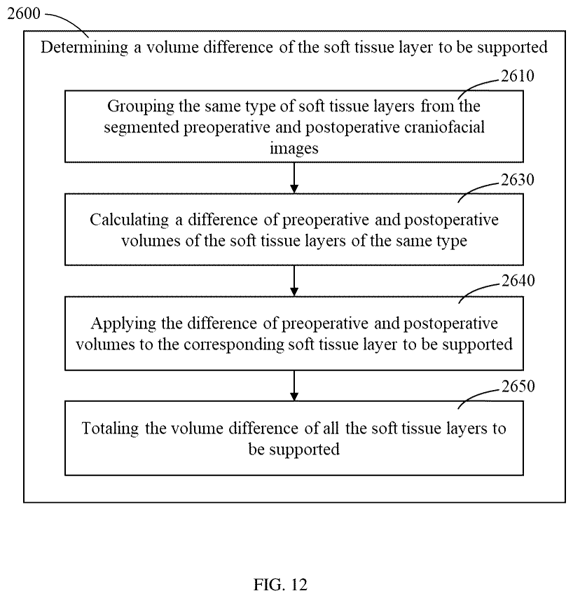

Next, FIG. 11 shows a schematic image representing normal paths from the base outline to a supported location, determined in accordance with the subcomponent step of matching the reference location with the supported location so as to form a normal path 2490, mentioned earlier in connection with FIG. 8. Particularly, FIG. 11 shows a preferred example of a simplified normal path diagram 2500 which pertains to a case of an implant having an open supportive edge to be applied to a damaged right cranium (i.e., a continuation from FIG. 10C or 10D, above). The normal path diagram 2500 further comprises lines representing a reference location 2510 and a supported location 2530. Points on the reference location 2510 are reference nodes 2512 (circle marks), and, likewise, points on the supported location 2530 are support nodes 2532 (square marks). Normal paths 2520 are paths projecting perpendicularly from the reference nodes 2512 to the respective support nodes 2532, thereby bridging the gaps between the reference locations 2510 and the supported locations 2530. It should be noted that the density of the nodes 2512, 2532 on their respective locations 2510, 2530 may be adjusted by a skilled person to achieve the optimal results according to the circumstances, and that the density shown in FIG. 11 has been simplified for the illustration's purpose and so does not represent the density of nodes in the actual implementation of the method, wherein the density of nodes would preferably be greater for designing a smaller implant, and vice versa.

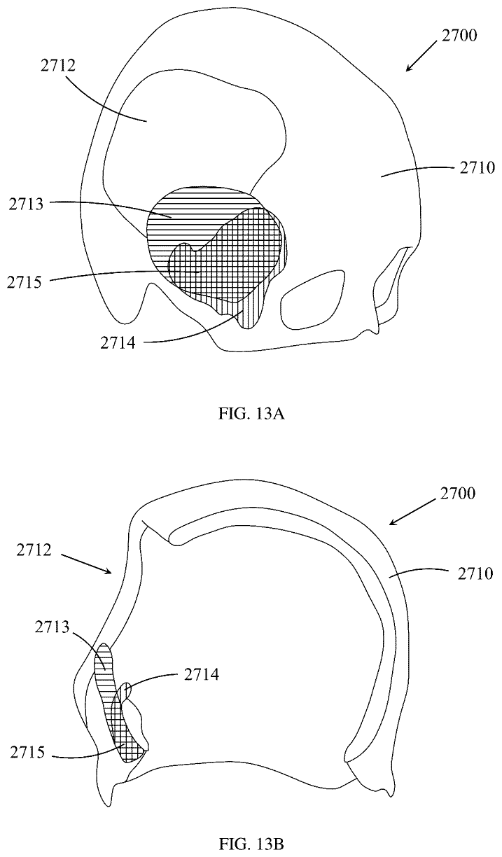

FIG. 12 shows a schematic flowchart representing the step of determining a volume difference of the soft tissue layer to be supported in accordance with a preferred embodiment. Here, the step of determining a volume difference of the soft tissue layer to be supported 2600, previously shown in FIG. 1, further comprises the following subcomponents: grouping the same type of soft tissue layers from the segmented preoperative and postoperative craniomaxillofacial images 2610; then, calculating a difference of preoperative and postoperative volumes of the soft tissue layers of the same type 2630; applying the difference of preoperative and postoperative volumes to the corresponding soft tissue layer to be supported 2640; and totaling the volume difference of all the soft tissue layers to be supported 2650. Said volume difference determined in accordance with these subcomponent steps provide the method in accordance with this preferred embodiment with a quantifiable constraint placed upon the total departures from the reference locations, thereby optimizing the support conferred by the implant being designed. The detail and products of some subcomponent steps as shown in FIG. 12 will be further illustrated in the following examples of FIGS. 13A and 13B.

FIG. 13A shows a perspective front view representing the difference between the preoperative and postoperative volumes of the soft tissue layer of the same type, calculated in accordance with a preferred embodiment. FIG. 13B shows a sectioned rear view representing the difference between the preoperative and postoperative volumes of the soft tissue layer of the same type, calculated in accordance with a preferred embodiment. Both FIGS. 13A and 13B show the different views of same craniomaxillofacial image 2700 having patient's craniomaxillofacial bone 2710 with a damaged right cranium 2712. FIGS. 13A and 13B further show a postoperative soft tissue layer 2713 and a preoperative soft tissue layer 2714, each of which has been obtained from the respective segmentation step 1600 as previously mentioned in FIG. 1. The postoperative soft tissue layer 2713 and the preoperative soft tissue layer 2714 as exemplified in FIGS. 13A and 13B are muscle layers as per the depiction in FIGS. 5A and 5B (i.e. a temporalis muscle). Visually overlapping the postoperative soft tissue layer 2713 and the preoperative soft tissue layer 2714 within common frame of reference produces a soft tissue intersection 2715. In such case, the volume difference in regard to the muscle layer as calculated from the respective subcomponent step 2630, would be the volumes of the postoperative soft tissue layer 2713 and the preoperative soft tissue layer 2714, excluding the volume of soft tissue intersection 2715.

Said volume difference is then applied to the corresponding muscle layer to be supported in accordance with the respective subcomponent step 2640. Subsequently, the same subcomponent steps 2630, 2640 are reiterated for the remaining soft tissue layers to be supported for the circumstances of operation (e.g. in the case of damaged right cranium as per FIGS. 13A and 13B, said remaining soft tissue layers to be supported would be fat and skin layers). The volume difference of all the soft tissue layers to be supported is then totaled in accordance with the respective subcomponent step 2650.

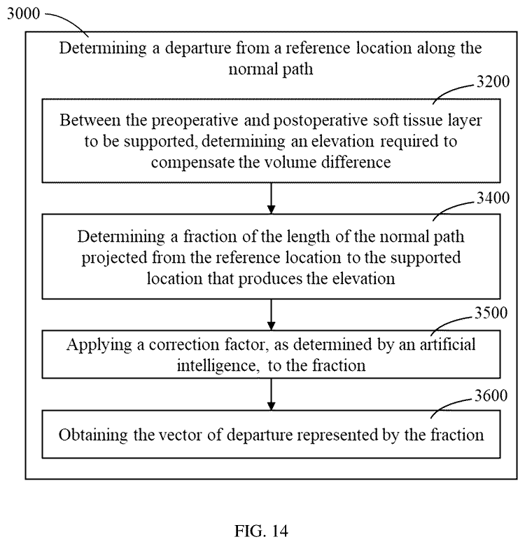

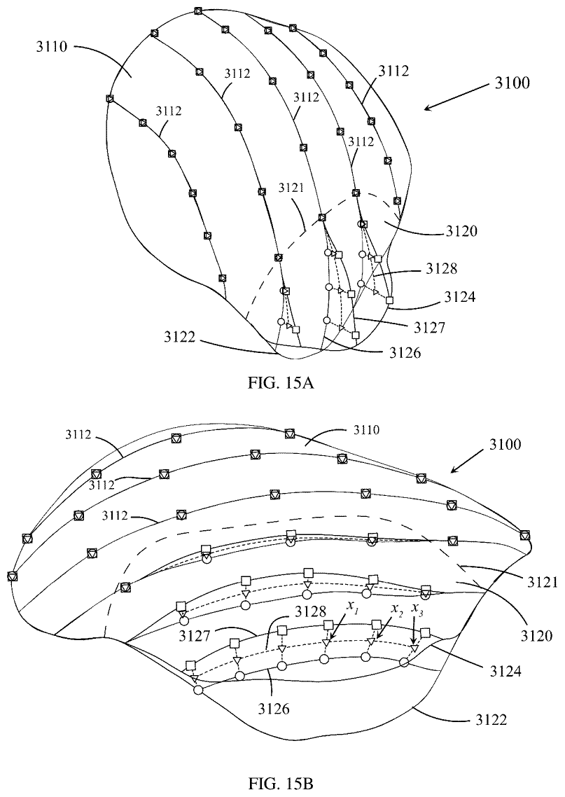

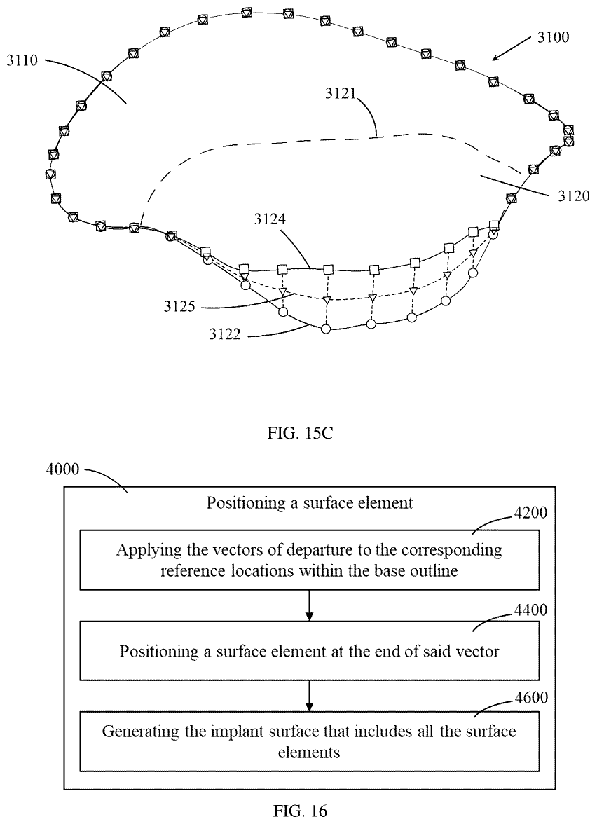

Next, FIG. 14 shows a schematic flowchart representing the step of determining a departure from a reference location along the normal path in accordance with a preferred embodiment. Here, the step of determining a departure from a reference location along the normal path 3000, previously shown in FIG. 1, further comprises the following subcomponents: between the preoperative and postoperative soft tissue layer to be supported, determining an elevation required to compensate the volume difference 3200; determining a fraction of the length of the normal path projected from the reference location to the supported location that produces the elevation 3400; applying a correlation factor, as determined by an artificial intelligence, to the fraction 3500; and then obtaining the vector of departure represented by the fraction 3600. The total volume difference as determined in accordance with the respective step 2600 is utilized here as a constraint for the total departures. The detail and products of the subcomponent steps in accordance with FIG. 14 are to be depicted in following FIGS. 15A, 15B, and 15C, all of which pertain to an exemplary embodiment wherein the implant being designed is for a reconstructive operation to be performed upon the patient's damaged right cranium.

In accordance with the preferred embodiment, FIG. 15A, 15B, and 15C show departures from the reference locations along the normal paths with respect to the first axis, the second axis, and the boundary of the implant being designed, respectively. It should be noted that, first, while the first and second axes in FIGS. 15A and 15B are perpendicularly disposed, such angular relationship may be adjusted by a skilled person to suit the circumstances of the operation and thus dose not limit the scope of this aspect of the present invention; second, as with the earlier part of the description in connection with FIG. 11, the density of reference nodes and support nodes as shown in FIGS. 15A, 15 B, and 15C are simplified for illustration purpose only and thus are not representative of the density of nodes in the actual implementation of the preferred embodiment of the method. The foregoing notes apply fully to the subsequent drawings pertaining to alternative embodiments directed to different craniomaxillofacial bones.

FIGS. 15A, 15B and 15C show an implant base outline 3100 which comprise elements of the boundary 3122, the surface constraints 3112, and the anatomic region of interest 3120. These three elements forming the base outline 3100 have been determined by the respective step 2200, previously depicted by FIGS. 6, 7A, and 7B and described accordingly. According to FIGS. 15A, 15B and 15C, the base outline 3100 is separated into two major zones: the baseline surface 3110 which embodies no departure, and the anatomic region of interest 3120 which potentially embodies the departure. These two major zones 3110, 3120 are visually separated by the divergent line 3121. For the illustration purpose, the divergent line 3121 also serves as the reference for different angles from which FIGS. 15A, 15B, and 15C are viewed.

Both FIGS. 15A and 15B further show a plurality of reference locations 3126 running preferably along the surface constraints 3112, a plurality of supported locations 3127 which are shown to superimpose upon the reference locations 3126 within the baseline surface 3110 and diverge from the reference locations 3126 within the anatomic region of interest 3120. FIGS. 15A and 15B also visualize the supported boundary 3124 which corresponds with the maximum possible elevation that would result from the combination of all the supported locations 3127. Between the two possible extremes indicated by the reference locations 3126 (i.e. 0% departure from the baseline surface 3110) and the supported locations 3127 (i.e. 100% departure from the baseline surface 3110), lies a plurality of departure locations 3128 (i.e. a fraction of x % departure from the baseline surface 3110, x being represented by a fraction between 0-100%). In a manner similar to FIG. 11 above, FIGS. 15A and 15B show a normal path projecting from each of the reference nodes (0% departure, o) along the reference locations 3126 to each of the support nodes (100% departure, a) along the supported locations 3127. Between each of said normal paths, the departure node (x % departure, A) indicates the actual departure from the baseline surface 3110 at that particular position. FIGS. 15A and 15B also show that the length of departure varies over a plurality of the reference locations 3126; and that the length of normal path varies over a plurality of the reference locations 3126. It also follows that, since the preferred embodiment as per FIGS. 15A and 15B takes into account the different patient-specific supports required over a plurality of reference locations 3126 within the anatomic region of interest 3120, the fraction representing x % departure of the departure locations 3128 is varied over a plurality of the reference locations 3126 as well. In accordance with the preferred embodiment, as shown in FIG. 14, wherein the step of applying a correlation factor, as determined by an artificial intelligence, to the fraction 3500, is employed, the fraction representing x % departure of the departure locations 3128 may be varied within the same departure location 3128. For example, said application of the correlation factor may cause x₁, x₂, and x₃, shown in FIG. 15B, which represent the departures at different departure nodes (Δ) within the same departure location 3128, to be of significantly different values.

FIG. 15C highlights a comparison between the boundary 3122, the supported boundary 3124, and the departure boundary 3125 at the edge of the anatomic region of interest 3120. In view of the foregoing FIGS. 15A and 15B, it should as well be emphasized that the departure and/or fraction of the normal path may vary throughout the anatomic region of interest 3120 depending on the patient-specific support required at particular positions, and so the departure/fraction exhibited by the departure boundary 3125 is not necessary the greatest within the anatomic region of interest 3120.

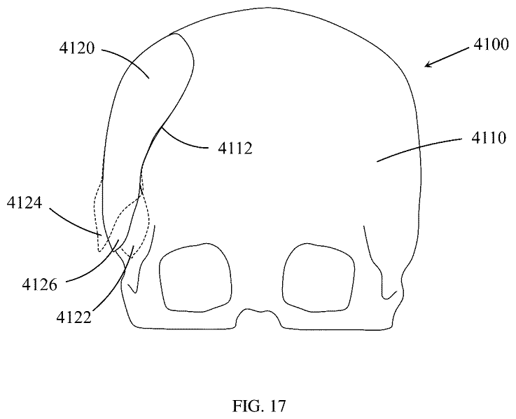

Furthermore, FIG. 16 shows a schematic flowchart representing the step of positioning a surface element in accordance with a preferred embodiment. Here, the step of positioning a surface element 4000, previously shown in FIG. 1, further comprises the following subcomponents: applying the vectors of departure to the corresponding reference locations within the base outline 4200; positioning a surface element at the end of said vector 4400; and finally, generating the implant surface that includes all the surface elements 4600. Examples of said surface element include a pixel or voxel which is part of the final surface of the implant being designed. Said vectors of departure start from their respective reference nodes and end at the departure nodes, disposed along their normal path towards the respective support nodes. Where there is no departure (e.g. a position within the baseline surface, or within the anatomic region of interest but no departure is assigned) the size of said departure vectors is zero, and the surface element is thus positioned upon the baseline surface. In this preferred embodiment, the first two subcomponent steps 4200, 4400 are reiterated upon all surface elements within the boundary until finally the implant surface is completely generated in accordance with the respective step 4600. The product and detail of the foregoing subcomponent steps are depicted by the following FIG. 17.

FIG. 17 shows an image of a cranial implant, the surface of which being formed by positioning the surface elements in accordance with a preferred embodiment. In particular, FIG. 17 shows a craniomaxillofacial image 4100 comprising a patient's craniomaxillofacial bone 4110 having a damaged right cranium 4112, to which an implant surface 4120 is visually applied. FIG. 17 further shows the possible range 4122, 4124 within the anatomic region of interest, within such range the implant surface 4120 may be positioned depending upon the patient-specific supports that have been transformed into the departure and/or fraction from the base outline as per fully described in connection with the above drawings. The minimum surface 4122 represents the case where no departure from the base outline has been determined (i.e. 0% departure throughout the implant surface), and so all the surface elements stay upon the base outline notwithstanding the anatomic region of interest; the maximum surface 4124 represents the case where all the departures within the anatomic region of interest coincide with the supported boundary (i.e. 100% departure throughout the anatomic region of interest). In this embodiment, following the step of positioning a surface element as per FIG. 16, all the surface elements are positioned so as to form an optimal surface 4126, disposed between the minimum surface 4122 and the maximum surface 4124.

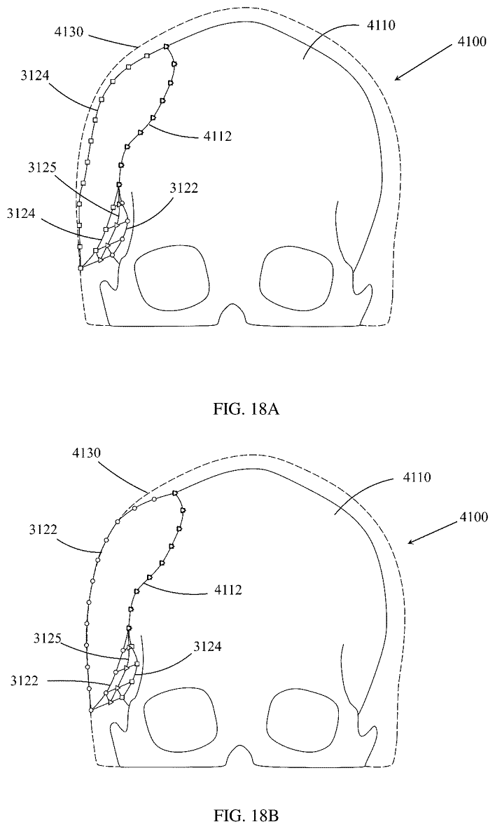

It should be noted further that the reference location does not always have to coincide with the layer of craniomaxillofacial bone. While the above description is directed to embodiments wherein the reference locations are located at the bone layer and the supported locations and departure locations represent the elevation from such bone layer to one of the overlaying soft tissue layers, such is not necessary to the methods according to the first aspect. The following FIGS. 18A and 18B shall shows exemplary embodiments which will illustrate such variations of form within the present inventive concept.

FIG. 18A shows an exemplary embodiment where the reference location coincides with a craniomaxillofacial bone. Alternatively, FIG. 18B shows another exemplary embodiment where the reference location coincides with a soft tissue layer. Both FIGS. 18A and 18B show a patient's craniomaxillofacial image 4100 comprising a craniomaxillofacial bone 4110 having a damaged right cranium 4112, and a soft tissue layer 4130 which in these embodiments is a skin layer. In FIG. 18A, the boundary 3122 having the reference location and the respective reference nodes (0% departure, o) coincides with the craniomaxillofacial bone 4110; the supported boundary 3124 having the supported locations and the respective supported nodes (100% departure, coincides with the skin layer 4130; and the departure boundary 3125 having the departure location and the respective departure nodes (x % departure, A) represents the elevations from the reference location (i.e. the craniomaxillofacial bone 4110) towards the supported location (i.e. the skin layer 3124). The direction of departures shown in FIG. 18A is in accordance with the above-described embodiments up to FIG. 17. On the other hand, FIG. 18B shows a reverse direction of such departures: The boundary 3122 having the reference location and the respective reference nodes (0% departure, o) coincides with the skin layer 4130; the supported boundary 3124 having the supported locations and the respective supported nodes (100% departure, u) coincides with the craniomaxillofacial bone 4110; and the departure boundary 3125 having the departure location and the respective departure nodes (x % departure, A) represents the descents from the reference location (i.e. the skin layer 4130) towards the supported location (i.e. the craniomaxillofacial bone 4110).

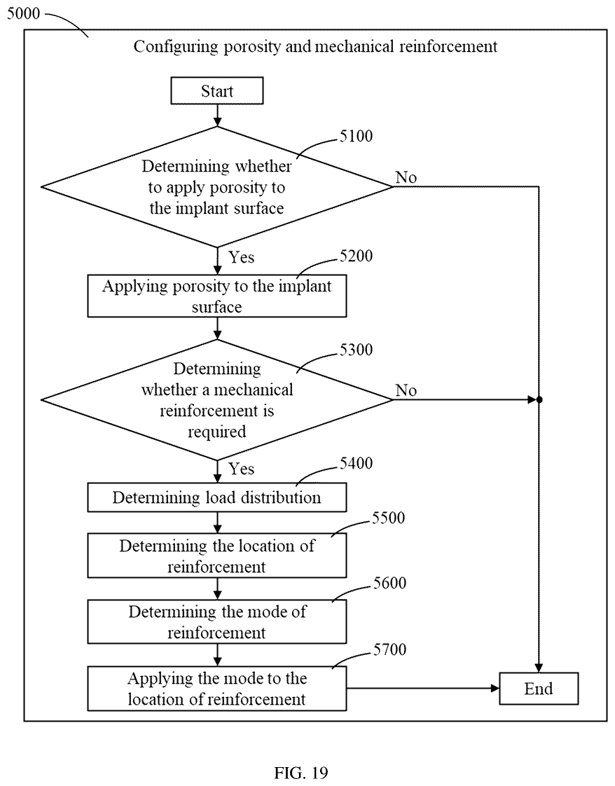

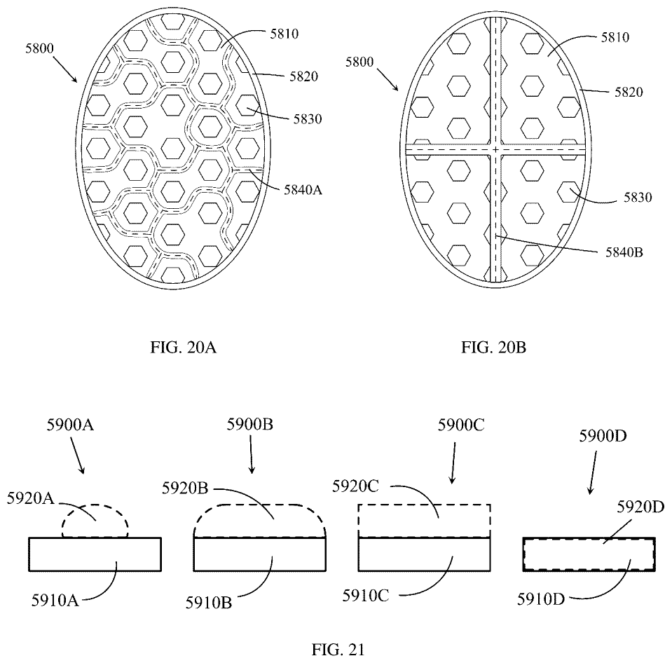

Next, FIG. 19 shows a schematic flowchart representing the step of configuring porosity and mechanical reinforcement in accordance with a preferred embodiment. Here, the step of configuring porosity and mechanical reinforcement 5000, previously shown in FIG. 1, further comprises the following subcomponents: determining whether to apply porosity to the implant surface 5100; if yes, the porosity is applied to the implant surface by the respective step 5200, and then then the method proceeds to determining whether a mechanical reinforcement is required 5300; if yes, the method proceeds to determining load distribution 5400 which will later be the basis of the later subcomponent steps of determining the location of reinforcement 5500, and determining the mode of reinforcement; after the foregoing elements of mechanical reinforcement have been determined by the respective subcomponent steps 5400, 5500, 5600, the method finally proceeds to applying the mode to the location of reinforcement 5700. Alternatively, the “no” decision at any of the respective subcomponent steps 5100, 5300 would cause the step of configuring porosity and mechanical reinforcement 5000 to end, thus the implant being designed would not embody the porosity and/or mechanical reinforcement without deviating from the scope of the present invention. The inclusion of porosity and mechanical reinforcement issues into a single decision loop is preferred by the present inventors. This is because in a circumstance that porosity is applied to the implant surface as required or preferred, such porosity would likely affect the mechanical strength of the final implant, and so the decision regarding the mechanical reinforcement should follow. It should be noted also that a mechanical reinforcement may be selectively applied depending on the operation's circumstantial requirements. For example, the reinforcement may be applied uniformly upon the implant surface, or non-uniformly based upon certain conditions. For further example, the reinforcement may be excluded from a specific locale upon the implant surface to accommodate manual adjustments which may be made mid-operation to that locale of the implant. The detail and products of the foregoing subcomponent steps are depicted by following FIGS. 20A, 20B, and 21.

Optionally, FIG. 20A shows a schematic image of an implant, the porosity and mechanical reinforcement of which being configured in accordance with the first alternative of a preferred embodiment. In this embodiment, the simplified implant 5800 comprises an implant surface 5810 and a boundary 5820. A porosity 5830, taking the form of uniformly distributed hexagonal holes, has been determined and applied to the implant surface 5810 in accordance with the respective subcomponent steps 5100, 5200. Furthermore, the first alternative of mechanical reinforcement 5840A, taking the form of uniformly distributed curved tracks which locally thicken the implant surface 5810 around the porosity 5810, has been determined and applied to the implant surface 5810 in accordance with the respective subcomponent steps 5300, 5400, 5500, 5600, and 5700.

Optionally, FIG. 20B shows a schematic image of an implant, the porosity and mechanical reinforcement of which being configured in accordance with the second alternative of a preferred embodiment. In this embodiment, the simplified implant 5800 comprises an implant surface 5810 and a boundary 5820. A porosity 5830, taking the form of uniformly distributed hexagonal holes, has been determined and applied to the implant surface 5810 in accordance with the respective subcomponent steps 5100, 5200. Furthermore, the second alternative of mechanical reinforcement 5840B, taking the form of two crossing straight bars, each running symmetrically across the implant surface 5810 and thickening the implant surface 5810 as well as partially blocking the porosity 5830, has been determined and applied to the implant surface 5810 in accordance with the respective subcomponent steps 5300, 5400, 5500, 5600, and 5700.

FIG. 21 shows variations of the modes of mechanical reinforcement of in accordance with a preferred embodiment. These are the example modes of mechanical reinforcement which may be determined by the respective subcomponent step 5600 to suit the circumstances of the operation and/or implant. Such examples, schematically depicted by cross-section views of the implant surface, include: a narrow track 5900A wherein an implant surface 5910A is thickened by augmenting a narrow reinforcement 5920A; a wide track 5900B wherein an implant surface 5910B is thickened by augmenting a wide reinforcement 5920B; a bar 5900C, wherein an implant surface 5910C is attached with a bar 5920C which may run over the porosity (not shown); and a filling 5900D, wherein an implant surface 5910D is not thickened, though a filling 5920D is applied to some of the porosity to provide the required mechanical reinforcement.

### Alternative Embodiments Directed to Other Craniomaxillofacial Bones

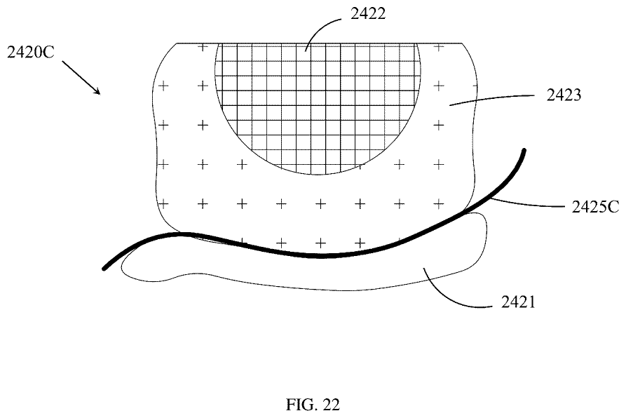

Next, the following FIGS. 22 to 28 show several alternative embodiments related to the first aspect (i.e. method of designing a craniomaxillofacial implant) of the present invention. Such alternative embodiments concern the application of the method to the design of craniomaxillofacial bones other than the cranium, which has already been addressed by the previous drawings and accompanying description. Said drawings shall also demonstrate that the present inventive concept may be applied fully to other craniomaxillofacial bones without deviating from the scope of the present invention. For brevity, it should be noted that following FIGS. 22 to 28 and their accompanying description will address only notable differences between the design of implants for cranium and for each of the other craniomaxillofacial bones. The unmentioned detail of the method and subcomponents thereof shall be considered to be substantially in accordance with the corresponding detail of the above embodiments as applied to the designing of a cranium implant.

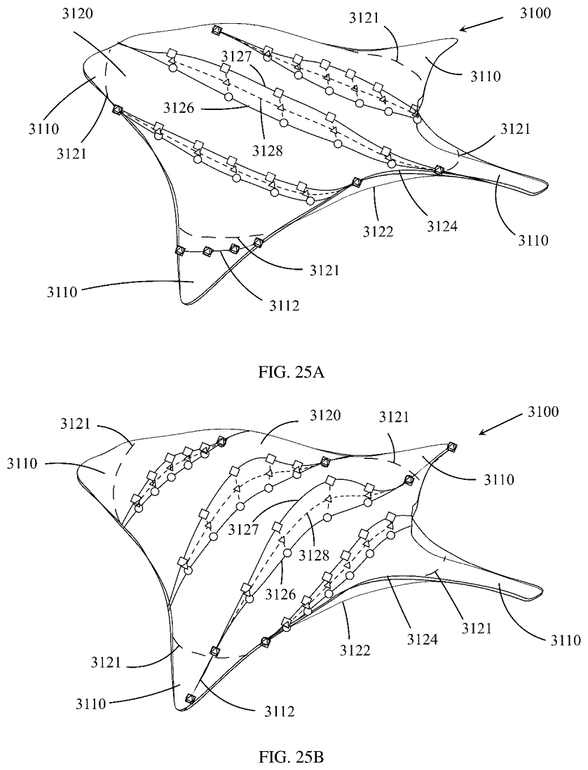

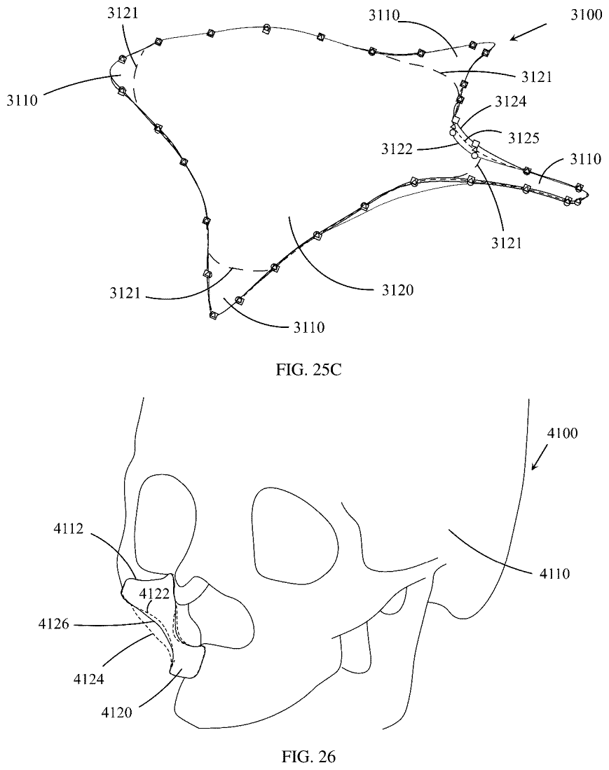

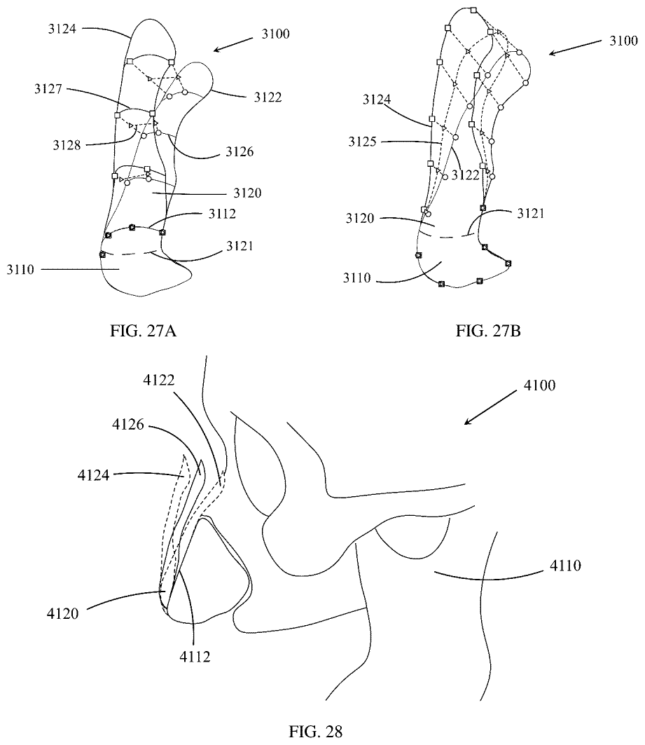

Specifically, FIGS. 22-24 illustrates the application of the embodiment to the design of orbital implant; FIGS. 25A-26 illustrates the application of the embodiment to the design of maxilla-zygomatic (i.e. midface) implant; and FIGS. 27A-28 illustrates the application of the embodiment to the design of nasal implant.

### Designing an Orbital Implant

FIG. 22 shows an assignment of the base outline in a scenario that the soft tissue layer to be supported includes only fat and muscle, in accordance with an alternative embodiment. Specifically, FIG. 22 depicts a scenario wherein the subcomponent steps of determining the soft tissue layer to be supported 2410; and assigning the base outline under, and adjacently to, the bottom of the innermost layer of the soft tissue layer to be supported 2430, as previously appeared in FIGS. 8, 9A, and 9B, are applied to the design of an orbital implant. Here, a cross-section postoperative image of the right orbit 2420C comprises, from the innermost to the outermost layer: a bone layer 2421, a fat layer 2423, and then a muscle layer 2422. It should be noted that, as opposed to a case of cranial bone, skin layer is not relevant to this operation; the fat layer 2423 lays closer to the bone layer 2421 than the muscle layer 2422 does; and, in the case of an orbit, the muscle layer 2422 is an eyeball. The soft tissue layer to be supported is determined by the respective subcomponent step to include both the muscle 2422 and fat 2423 layers; thus, the base outline 2425C is assigned by the respective subcomponent step to be under, and adjacently to, the bottom of the innermost layer of the soft tissue to be supported, which in this case is the bottom of the fat layer 2423. It is also to be appreciated that if the implant being designed were to depart from the base outline 2425C, such departure would affect the support imparted to the fat layer 2423 only. This is because, as previously noted, a skin layer is not relevant, and the muscle 2422 layer is an eyeball.

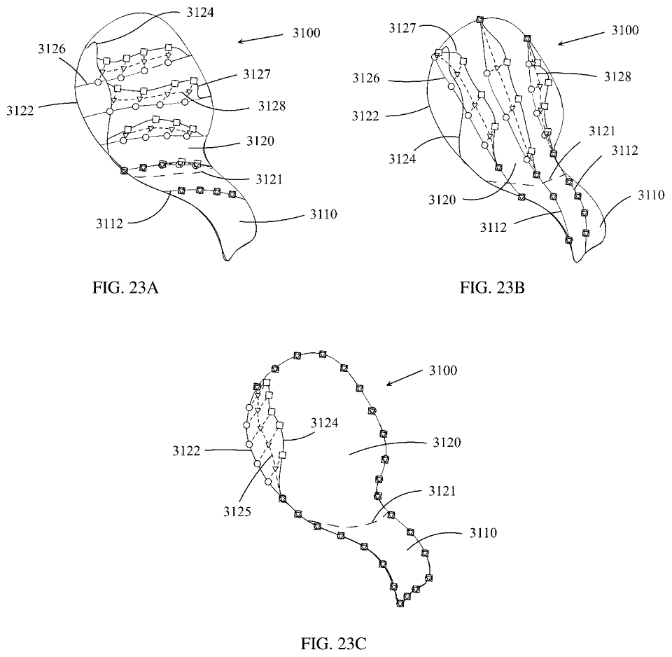

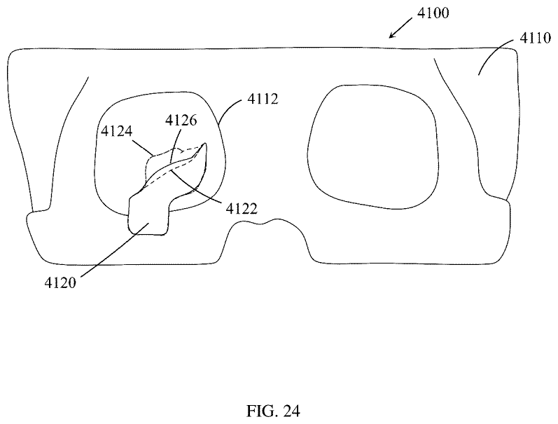

Next, FIGS. 23A, 23B and 23C show an implant base outline 3100 which comprise elements of the boundary 3122, the surface constraints 3112, and the anatomic region of interest 3120. These three elements forming the base outline 3100 have been determined by the respective step 2200, previously depicted by FIGS. 6, 7A, and 7B and described accordingly. Specifically, FIGS. 23A, 23B and 23C depict a scenario wherein the subcomponent steps of determining a departure from a reference location along the normal path 3000, as previously appeared in FIGS. 14, 15A, 15B, and 15C, are applied to designing an orbital implant.

More specifically, according to FIGS. 23A, 23B and 23C, the base outline 3100 is also separated into two major zones: the baseline surface 3110 which embodies no departure, and the anatomic region of interest 3120 which potentially embodies the departure. These two major zones 3110, 3120 are visually separated by the divergent line 3121.

Both FIGS. 23A and 23B further show a plurality of reference locations 3126 running preferably along the surface constraints 3112, a plurality of supported locations 3127 which are shown to superimpose upon the reference locations 3126 within the baseline surface 3110 and diverge from the reference locations 3126 within the anatomic region of interest 3120. FIGS. 23A and 23B also visualize the supported boundary 3124 which corresponds with the maximum possible elevation that would result from the combination of all the supported locations 3127. Between the two possible extremes indicated by the reference locations 3126 (i.e. 0% departure from the baseline surface 3110) and the supported locations 3127 (i.e. 100% departure from the baseline surface 3110), lies a plurality of departure locations 3128 (i.e. a fraction of x % departure from the baseline surface 3110, x being represented by a fraction between 0-100%). FIGS. 23A and 23B further show a normal path projecting from each of the reference nodes (0% departure, o) along the reference locations 3126 to each of the support nodes (100% departure, a) along the supported locations 3127. Between each of said normal paths, the departure node (x % departure, A) indicates the actual departure from the baseline surface 3110 at that particular position. FIGS. 23A and 23B also show that the length of departure varies over a plurality of the reference locations 3126; and that the length of normal path varies over a plurality of the reference locations 3126. It also follows that, since the alternative embodiment as per FIGS. 23A and 23B takes into account the different patient-specific supports required over a plurality of reference locations 3126 within the anatomic region of interest 3120, the fraction representing x % departure of the departure locations 3128 is varied over a plurality of the reference locations 3126 as well.

FIG. 23C highlights a comparison between the boundary 3122, the supported boundary 3124, and the departure boundary 3125 at the edge of the anatomic region of interest 3120. In view of the foregoing FIGS. 23A and 23B, it should as well be emphasized that the departure and/or fraction of the normal path may vary throughout the anatomic region of interest 3120 depending on the patient-specific support required at particular positions, and so the departure/fraction exhibited by the departure boundary 3125 is not necessary the greatest within the anatomic region of interest 3120.

FIG. 24 shows an image of an orbital implant, the surface of which being formed by positioning the surface elements in accordance with an alternative embodiment. Specifically, FIG. 24 depicts a scenario wherein the subcomponent steps of positioning a surface element 4000, as previously appeared in FIGS. 16 and 17, are applied to designing an orbital implant.

FIG. 24 shows an image of an orbital implant, the surface of which being formed by positioning the surface elements in accordance with an alternative embodiment. In particular, FIG. 24 shows a craniomaxillofacial image 4100 comprising a patient's craniomaxillofacial bone 4110 having a damaged right orbit 4112, to which an implant surface 4120 is visually applied. FIG. 24 further shows the possible range 4122, 4124 within the anatomic region of interest, within such range the implant surface 4120 may be positioned depending upon the patient-specific supports that have been transformed into the departure and/or fraction from the base outline as per fully described in connection with the above drawings. The minimum surface 4122 represents the case where no departure from the base outline has been determined (i.e. 0% departure throughout the implant surface), and so all the surface elements stay upon the base outline notwithstanding the anatomic region of interest; the maximum surface 4124 represents the case where all the departures within the anatomic region of interest coincide with the supported boundary (i.e. 100% departure throughout the anatomic region of interest). In this embodiment, following the step of positioning a surface element as per FIG. 16 above, all the surface elements are positioned so as to form an optimal surface 4126, disposed between the minimum surface 4122 and the maximum surface 4124.

Designing a Maxilla-Zygomatic (i.e. Midface) Implant

FIGS. 25A, 25B and 25C show an implant base outline 3100 which comprise elements of the boundary 3122, the surface constraints 3112, and the anatomic region of interest 3120. These three elements forming the base outline 3100 have been determined by the respective step 2200, previously depicted by FIGS. 6, 7A, and 7B and described accordingly. Specifically, FIGS. 25A, 25B and 25C depict a scenario wherein the subcomponent steps of determining a departure from a reference location along the normal path 3000, as previously appeared in FIGS. 14, 15A, 15B, and 15C, are applied to designing a maxilla-zygomatic implant.

More specifically, according to FIGS. 25A, 25B and 25C, the base outline 3100 is also separated into two major zones: the baseline surface 3110 which embodies no departure, and the anatomic region of interest 3120 which potentially embodies the departure. These two major zones 3110, 3120 are visually separated by the divergent line 3121. Circumstantially, the anatomic region of interest 3120 in this alternative embodiment is located in the middle of four separated baseline surfaces 3110.

Both FIGS. 25A and 25B further show a plurality of reference locations 3126 running preferably along the surface constraints 3112, a plurality of supported locations 3127 which are shown to superimpose upon the reference locations 3126 within the baseline surface 3110 and diverge from the reference locations 3126 within the anatomic region of interest 3120. FIGS. 25A and 25B also visualize the supported boundary 3124 which corresponds with the maximum possible elevation that would result from the combination of all the supported locations 3127. Between the two possible extremes indicated by the reference locations 3126 (i.e. 0% departure from the baseline surface 3110) and the supported locations 3127 (i.e. 100% departure from the baseline surface 3110), lies a plurality of departure locations 3128 (i.e. a fraction of x % departure from the baseline surface 3110, x being represented by a fraction between 0-100%). FIGS. 25A and 25B further show a normal path projecting from each of the reference nodes (0% departure, o) along the reference locations 3126 to each of the support nodes (100% departure, o) along the supported locations 3127. Between each of said normal paths, the departure node (x % departure, A) indicates the actual departure from the baseline surface 3110 at that particular position. FIGS. 25A and 25B also show that the length of departure varies over a plurality of the reference locations 3126; and that the length of normal path varies over a plurality of the reference locations 3126. It also follows that, since the alternative embodiment as per FIGS. 25A and 25B takes into account the different patient-specific supports required over a plurality of reference locations 3126 within the anatomic region of interest 3120, the fraction representing x % departure of the departure locations 3128 is varied over a plurality of the reference locations 3126 as well.

FIG. 25C highlights a comparison between the boundary 3122, the supported boundary 3124, and the departure boundary 3125 at one edge of the anatomic region of interest 3120. In view of the foregoing FIGS. 25A and 25B, it should as well be emphasized that the departure and/or fraction of the normal path may vary throughout the anatomic region of interest 3120 depending on the patient-specific support required at particular positions, and so the departure/fraction exhibited by the departure boundary 3125 is not necessary the greatest within the anatomic region of interest 3120.

FIG. 26 shows an image of a maxilla-zygomatic implant, the surface of which being formed by positioning the surface elements in accordance with an alternative embodiment. Specifically, FIG. 26 depicts a scenario wherein the subcomponent steps of positioning a surface element 4000, as previously appeared in FIGS. 16 and 17, are applied to designing a maxilla-zygomatic implant.

FIG. 26 shows an image of an implant, the surface of which being formed by positioning the surface elements in accordance with an alternative embodiment. In particular, FIG. 26 shows a craniomaxillofacial image 4100 comprising a patient's craniomaxillofacial bone 4110 having a damaged right maxilla-zygomatic bone 4112, to which an implant surface 4120 is visually applied. FIG. 26 further shows the possible range 4122, 4124 within the anatomic region of interest, within such range the implant surface 4120 may be positioned depending upon the patient-specific supports that have been transformed into the departure and/or fraction from the base outline as per fully described in connection with the above drawings. The minimum surface 4122 represents the case where no departure from the base outline has been determined (i.e. 0% departure throughout the implant surface), and so all the surface elements stay upon the base outline notwithstanding the anatomic region of interest; the maximum surface 4124 represents the case where all the departures within the anatomic region of interest coincide with the supported boundary (i.e. 100% departure throughout the anatomic region of interest). In this embodiment, following the step of positioning a surface element as per FIG. 16 above, all the surface elements are positioned so as to form an optimal surface 4126, disposed between the minimum surface 4122 and the maximum surface 4124.

### Designing a Nasal Implant

FIGS. 27A and 27B shows an implant base outline 3100 which comprises elements of the boundary 3122, the surface constraints 3112, and the anatomic region of interest 3120. These three elements forming the base outline 3100 have been determined by the respective step 2200, previously depicted by FIGS. 6, 7A, and 7B and described accordingly. Specifically, FIGS. 27A and 27B depict a scenario wherein the subcomponent steps of determining a departure from a reference location along the normal path 3000, as previously appeared in FIGS. 14, 15A, 15B, and 15C, are applied to designing a nasal implant.

More specifically, according to FIG. 27A, the base outline 3100 is also separated into two major zones: the baseline surface 3110 which embodies no departure, and the anatomic region of interest 3120 which potentially embodies the departure. These two major zones 3110, 3120 are visually separated by the divergent line 3121.

FIG. 27A further shows a plurality of reference locations 3126 running preferably along the surface constraints 3112, a plurality of supported locations 3127 which are shown to superimpose upon the reference locations 3126 within the baseline surface 3110 and diverge from the reference locations 3126 within the anatomic region of interest 3120. FIG. 27A also visualizes the supported boundary 3124 which corresponds with the maximum possible elevation that would result from the combination of all the supported locations 3127. Between the two possible extremes indicated by the reference locations 3126 (i.e. 0% departure from the baseline surface 3110) and the supported locations 3127 (i.e. 100% departure from the baseline surface 3110), lies a plurality of departure locations 3128 (i.e. a fraction of x % departure from the baseline surface 3110, x being represented by a fraction between 0-100%). FIG. 27A further shows a normal path projecting from each of the reference nodes (0% departure, o) along the reference locations 3126 to each of the support nodes (100% departure, o) along the supported locations 3127. Between each of said normal paths, the departure node (x % departure, A) indicates the actual departure from the baseline surface 3110 at that particular position. FIG. 27A also shows that the length of departure varies over a plurality of the reference locations 3126; and that the length of normal path varies over a plurality of the reference locations 3126. It also follows that, since the alternative embodiment as per FIG. 27A takes into account the different patient-specific supports required over a plurality of reference locations 3126 within the anatomic region of interest 3120, the fraction representing x % departure of the departure locations 3128 is varied over a plurality of the reference locations 3126 as well.

FIG. 27B highlights a comparison between the boundary 3122, the supported boundary 3124, and the departure boundary 3125 within the anatomic region of interest 3120. In view of the foregoing FIG. 27B, it should as well be emphasized that the departure and/or fraction of the normal path may vary throughout the anatomic region of interest 3120 depending on the patient-specific support required at particular positions, and so the departure/fraction exhibited by the departure boundary 3125 is not necessary the greatest within the anatomic region of interest 3120.

FIG. 28 shows an image of a nasal implant, the surface of which being formed by positioning the surface elements in accordance with an alternative embodiment. Specifically, FIG. 28 depicts a scenario wherein the subcomponent steps of positioning a surface element 4000, as previously appeared in FIGS. 16 and 17, are applied to designing a nasal implant.

FIG. 28 shows an image of a nasal implant, the surface of which being formed by positioning the surface elements in accordance with an alternative embodiment. In particular, FIG. 28 shows a craniomaxillofacial image 4100 comprising a patient's craniomaxillofacial bone 4110 a nasal bone 4112 (in the context of the present description, the nasal bone is inclusive of nasal cartilage) upon which a cosmetic surgery is to be performed by way of applying a nasal implant having an implant surface 4120 visualized accordingly. FIG. 28 further shows the possible range 4122, 4124 within the anatomic region of interest, within such range the implant surface 4120 may be positioned depending upon the patient-specific supports that have been transformed into the departure and/or fraction from the base outline as per fully described in connection with the above drawings. The minimum surface 4122 represents the case where no departure from the base outline has been determined (i.e. 0% departure throughout the implant surface), and so all the surface elements stay upon the base outline notwithstanding the anatomic region of interest; the maximum surface 4124 represents the case where all the departures within the anatomic region of interest coincide with the supported boundary (i.e. 100% departure throughout the anatomic region of interest). In this embodiment, following the step of positioning a surface element as per FIG. 16 above, all the surface elements are positioned so as to form an optimal surface 4126, disposed between the minimum surface 4122 and the maximum surface 4124.

### Craniomaxillofacial Implant

The second aspect of the present invention is directed to a novel craniomaxillofacial implant enabled by the designing method in accordance with the first aspect. It should be noted that the implants' subcomponents as shown in the following drawings and mentioned in the following description may be modified by a relevant skilled person upon full knowledge of the Detailed Description and drawings.

### A Cranial Implant

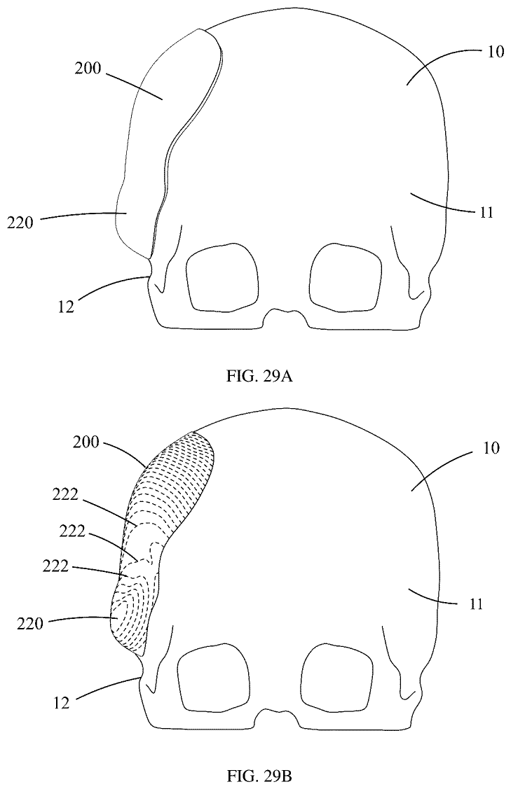

FIG. 29A schematically shows the application of a cranial implant in accordance with a preferred embodiment. In this embodiment, the craniomaxillofacial implant takes the form of a cranial implant 200 to be applied to a patient's craniomaxillofacial bone 10, specifically to a damaged right cranium 12. Said cranial implant 200 is formed, in accordance with the design enabled by the first aspect, into a plate that is rigid, warped, and of a substantially uniform thickness. The warpage 220 is configured preoperatively so as to adapt the cranial implant 200 to be set onto, and supported by, at least a craniomaxillofacial bone (i.e. the right cranium 12); and support the soft tissue layer (not shown) in place of at least said craniomaxillofacial bone, thereby compensating or augmenting the elevation of the soft tissue layer. Example scenarios wherein the warpage 220 is configured preoperatively so as to adapt the cranial implant 200 to also be set onto, and supported by a soft tissue layer overlaying the craniomaxillofacial bone, is illustrated as per the above FIG. 10B, wherein the resulting implant would be supported by a muscle layer. The subsequent FIG. 29B further shows that the warpage 220, as outlined by the topology mappings 222 is non-uniformly distributed upon the implant 200. According to both FIGS. 29A and 29B, the support provided by the warpage 220 to the soft tissue layer is asymmetrical to that provided by a contralateral portion 11 of the craniomaxillofacial bone 10.

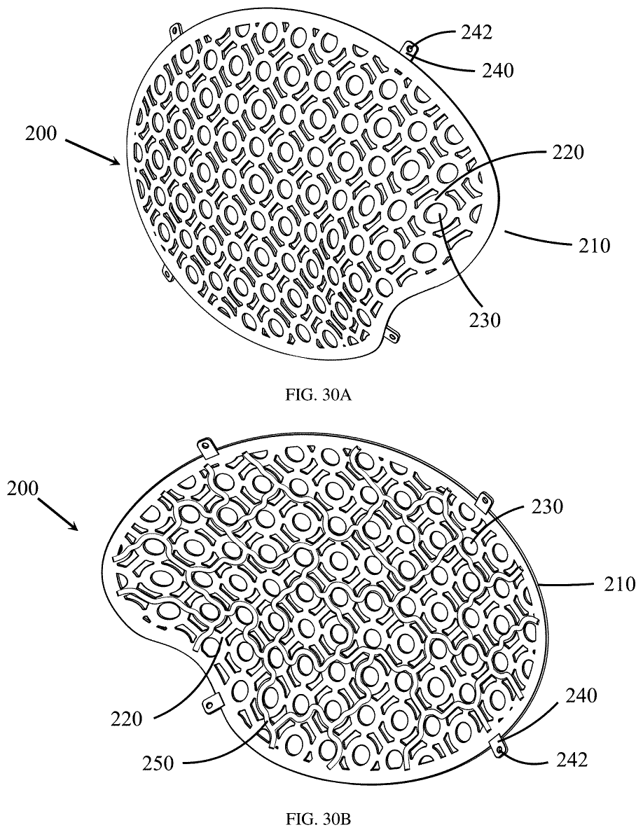

FIG. 30A shows a front perspective view of a cranial implant in accordance with a preferred embodiment. In this embodiment, the cranial implant 200 is made of a titanium alloy, formed into a rigid plate of a substantially uniform thickness which is within a range of 0.2-0.8 mm. Said range of thickness is also an important novel feature enabled by the first aspect. As with the previous FIGS. 29A and 29B, the cranial implant 200 comprises a warpage 220 that is non-uniformly distributed upon the implant 200. FIG. 30A also shows the cranial implant 200 to comprise further a porosity 230 which takes the form of uniformly distributed circular holes, each of said holes being connected by a plurality of narrow struts; and preferably four means for fastening 240 the cranial implant 200 upon the craniomaxillofacial bone. It should be noted also that the form of porosity may also be configured to suit the circumstantial needs of operation, e.g. the required strength and ventilation. In this embodiment, each of said means for fastening 240 takes the form of a fin protruding laterally outward from the cranial implant's 200 boundary 210. Said means for fastening 240 features a screw hole 242 for insertion of a screw (not shown). From a rear perspective view of FIG. 30B, the cranial implant 200 further comprises a mechanical reinforcement 250 that takes the form of a uniformly distributed curved tracks which locally thicken the implant surface around the porosity 230.

### An Orbital Implant

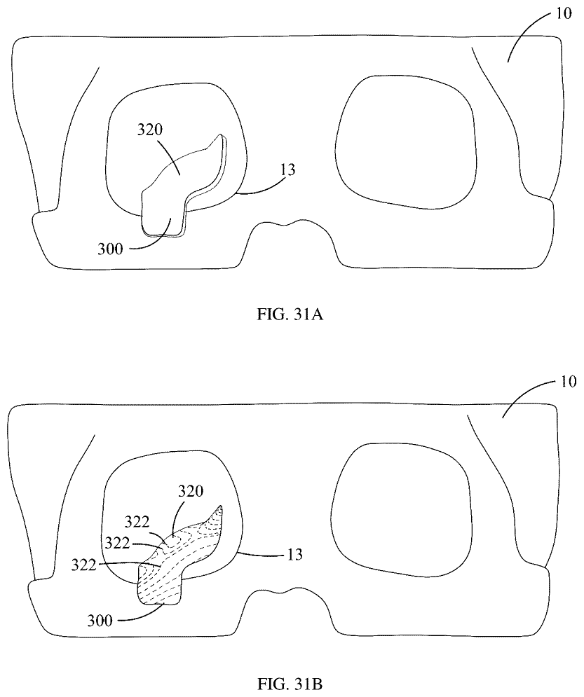

FIG. 31A schematically shows the application of an orbital implant in accordance with a preferred embodiment. In this embodiment, the craniomaxillofacial implant takes the form of an orbital implant 300 to be applied to a patient's craniomaxillofacial bone 10, specifically to a damaged right orbit 13. Said orbital implant 300 is formed, in accordance with the design enabled by the first aspect, into a plate that is rigid, warped, and of a substantially uniform thickness. The warpage 320 is configured preoperatively so as to adapt the orbital implant 300 to be set onto, and supported by, at least a craniomaxillofacial bone (i.e. the right orbit 13); and support the soft tissue layer (not shown) in place of at least said craniomaxillofacial bone, thereby compensating or augmenting the elevation of the soft tissue layer. The subsequent FIG. 31B further shows that the warpage 320, as outlined by the topology mappings 322 is non-uniformly distributed upon the implant 300. The support provided by the warpage 320 to the soft tissue layer is asymmetrical to that provided by a contralateral portion of the craniomaxillofacial bone.

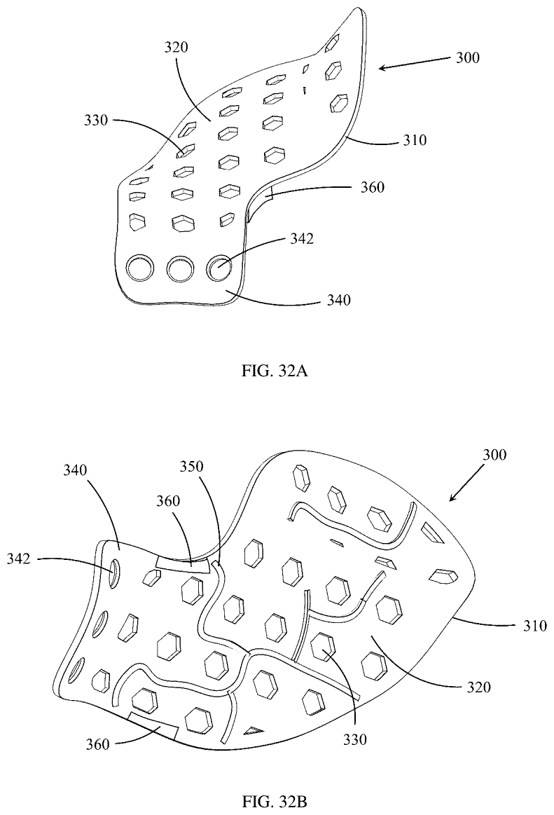

FIG. 32A shows a front perspective view of an orbital implant in accordance with a preferred embodiment. In this embodiment, the orbital implant 300 is made of a titanium alloy, formed into a rigid plate of a substantially uniform thickness which is within a range of 0.2-0.8 mm. Said range of thickness, which is also an important novel feature enabled by the first aspect, is particularly beneficial when the implant is for an orbit which is more sensitive to invasiveness than a cranium. As with the previous FIGS. 31A and 31B, the orbital implant 300 comprises a warpage 320 that is non-uniformly distributed upon the implant 300. FIG. 32A also shows the orbital implant 300 to comprise further a porosity 330 which takes the form of uniformly distributed hexagonal holes; and preferably a means for fastening 340 the orbital implant 300 upon the craniomaxillofacial bone. In this embodiment, the means for fastening 340 is located within the orbital implant's 300 boundary 310. Said means for fastening 340 features three screw holes 342, each for insertion of a screw (not shown). In this embodiment where the nature of an orbital bone requires additional stability of the implant 300, the means for fastening 340 is also adapted to bend, providing an extra surface to adjoin the lower edge of the orbit as shown earlier in FIGS. 31A and 31B, and thereby increasing stability. For the same reason, this embodiment also comprises a ridge 360 adapted to further increase the stability of the orbital implant 300 when set onto, and supported by, the craniomaxillofacial bone. From a rear perspective view of FIG. 32B, the orbital implant 300 comprises two of such ridges 360, and also a mechanical reinforcement 350 that takes the form of a non-uniformly distributed curved tracks which locally thicken the implant surface around the porosity 330.

A Maxilla-Zygomatic (i.e. Midface) Implant

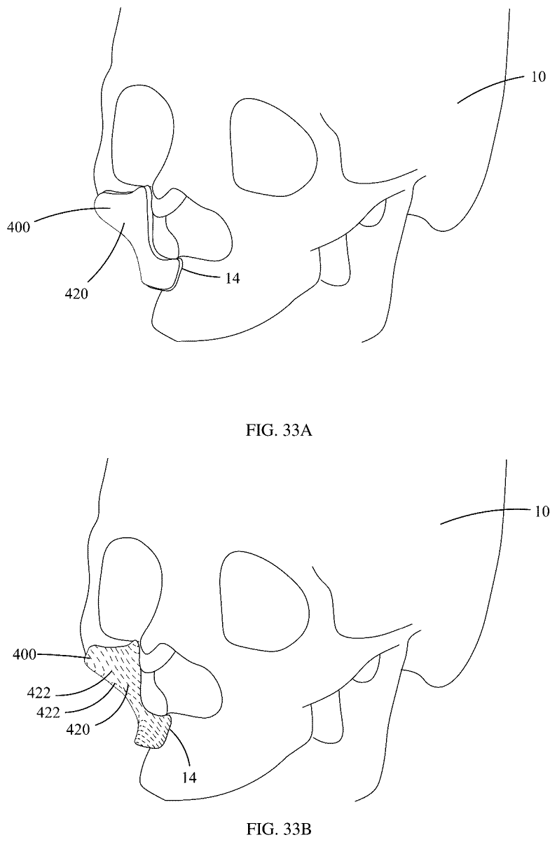

FIG. 33A schematically shows the application of a maxilla-zygomatic implant in accordance with a preferred embodiment. In this embodiment, the craniomaxillofacial implant takes the form of a maxilla-zygomatic implant 400 to be applied to a patient's craniomaxillofacial bone 10, specifically to a damaged right maxilla-zygomatic bone 14. Said maxilla-zygomatic implant 400 is formed, in accordance with the design enabled by the first aspect, into a plate that is rigid, warped, and of a substantially uniform thickness. The warpage 420 is configured preoperatively so as to adapt the maxilla-zygomatic implant 400 to be set onto, and supported by, at least a craniomaxillofacial bone (i.e. the right maxilla-zygomatic bone 14); and support the soft tissue layer (not shown) in place of at least said craniomaxillofacial bone, thereby compensating or augmenting the elevation of the soft tissue layer. The subsequent FIG. 33B further shows that the warpage 420, as outlined by the topology mappings 422 is non-uniformly distributed upon the implant 400. The support provided by the warpage 420 to the soft tissue layer is asymmetrical to that provided by a contralateral portion of the craniomaxillofacial bone.

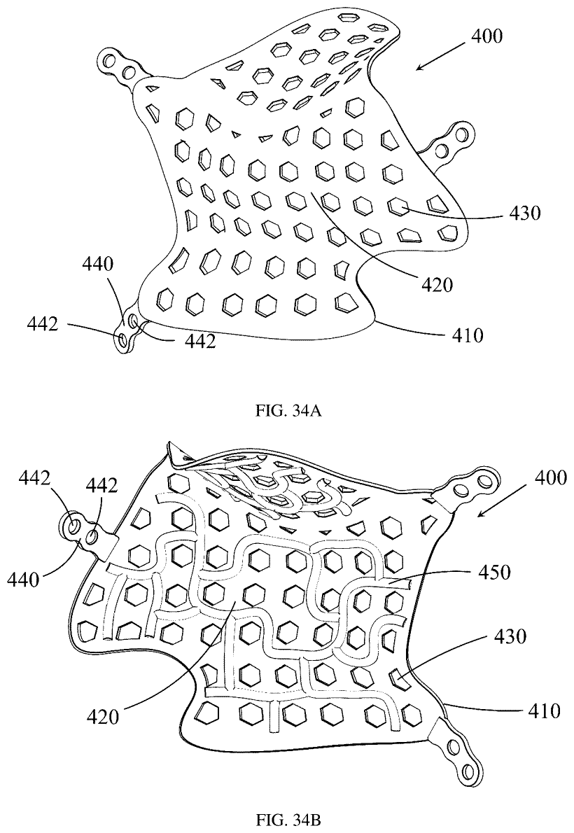

FIG. 34A shows a front perspective view of a maxilla-zygomatic implant in accordance with a preferred embodiment. In this embodiment, the maxilla-zygomatic implant 400 is made of a titanium alloy, formed into a rigid plate of a substantially uniform thickness which is within a range of 0.2-0.8 mm. Said range of thickness is also an important novel feature enabled by the first aspect. As with the previous FIGS. 33A and 33B, the maxilla-zygomatic implant 400 comprises a warpage 420 that is non-uniformly distributed upon the implant 400. FIG. 34A also shows the maxilla-zygomatic implant 400 to comprise further a porosity 430 which takes the form of uniformly distributed hexagonal holes; and preferably three means for fastening 440 the maxilla-zygomatic implant 400 upon the craniomaxillofacial bone. In this embodiment, each of said means for fastening 440 takes the form of a fin protruding laterally outward from the maxilla-zygomatic implant's 400 boundary 410. Said means for fastening 440 features two screw holes 442, each for insertion of a screw (not shown). From a rear perspective view of FIG. 34B, the maxilla-zygomatic implant 400 further comprises a mechanical reinforcement 450 that takes the form of a non-uniformly distributed curved tracks which locally thicken the implant surface around the porosity 430.

### A Nasal Implant

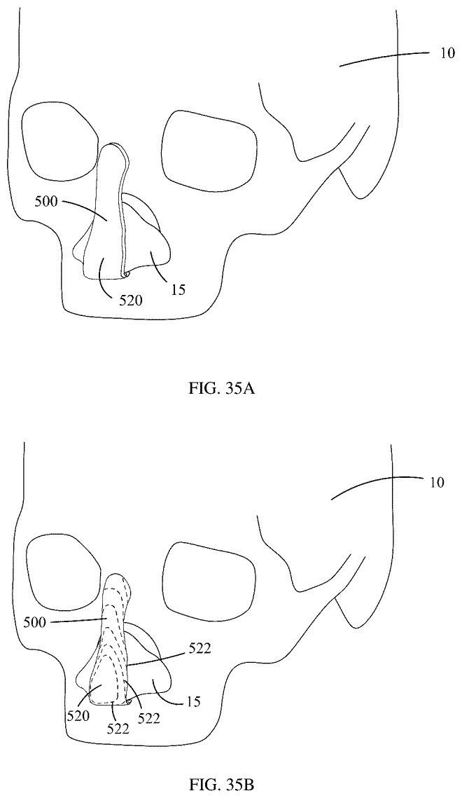

FIG. 35A schematically shows the application of a nasal implant in accordance with a preferred embodiment. In this embodiment, the craniomaxillofacial implant takes the form of a nasal implant 500 to be applied to a patient's craniomaxillofacial bone 10, specifically to a nasal bone 15 (in the context of the present description, the nasal bone is inclusive of nasal cartilage) upon which a cosmetic surgery is to be performed. Said nasal implant 500 is formed, in accordance with the design enabled by the first aspect, into a plate that is rigid, warped, and of a substantially uniform thickness. The warpage 520 is configured preoperatively so as to adapt the nasal implant 500 to be set onto, and supported by, at least a craniomaxillofacial bone (i.e. the nasal bone 15); and support the soft tissue layer (not shown) in place of at least said craniomaxillofacial bone, thereby compensating or augmenting the elevation of the soft tissue layer. The subsequent FIG. 35B further shows that the warpage 520, as outlined by the topology mappings 522 is uniformly distributed upon the implant 500.

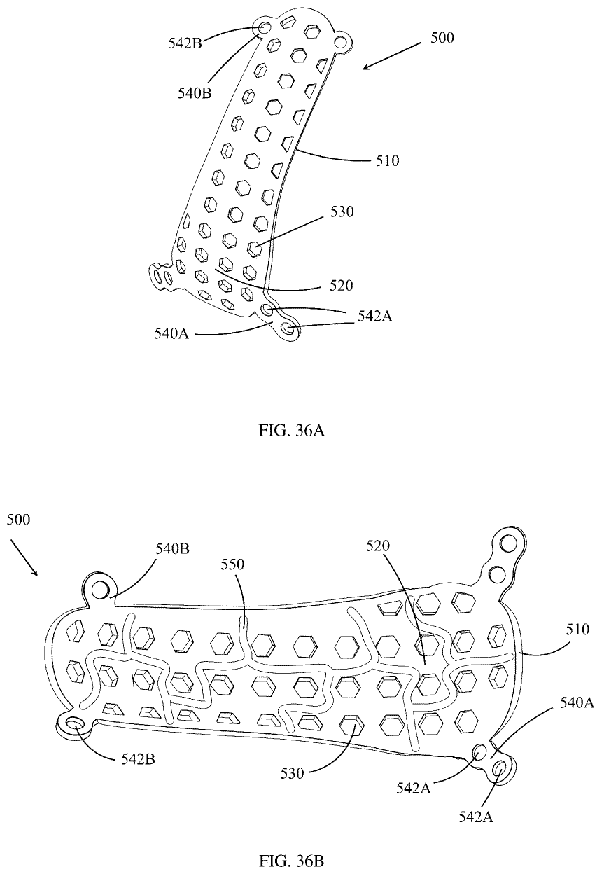

FIG. 36A shows a front perspective view of a nasal implant in accordance with a preferred embodiment. In this embodiment, the nasal implant 500 is made of silicone, formed into a rigid plate of a substantially uniform thickness which is within a range of 0.2-0.8 mm. Said range of thickness, which is also an important novel feature enabled by the first aspect, is particularly beneficial when the implant is for a nasal bone which is more sensitive to invasiveness than a cranium. As with the previous FIGS. 35A and 35B, the nasal implant 500 comprises a warpage 520 that is uniformly distributed upon the implant 500. FIG. 36A also shows the nasal implant 500 to comprise further a porosity 530 which takes the form of uniformly distributed hexagonal holes; and preferably two of a first means for fastening 540A and two of a second means for fastening 540B, both for fastening the nasal implant 500 upon the craniomaxillofacial bone. In this embodiment, each of the first means for fastening 540A takes the form of a fin protruding laterally outward from the nasal implant's 500 boundary 510, each of said first means for fastening 540A also features two screw holes 542A, each for insertion of a screw (not shown). Further, each of the second means for fastening 540B takes the form of a fin protruding laterally outward from the nasal implant's 500 boundary 510, each of said second means for fastening 540B also features one screw hole 542B for insertion of a screw (not shown). From a rear perspective view of FIG. 36B, the nasal implant 500 further comprises a mechanical reinforcement 550 that takes the form of a uniformly distributed curved tracks which locally thicken the implant surface around the porosity 530.

### A Mandibular Implant

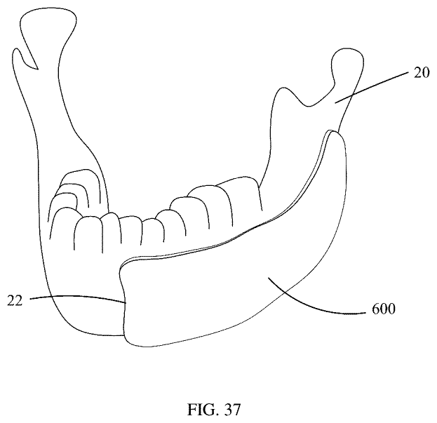

FIG. 37 schematically shows the application of a mandibular implant in accordance with a preferred embodiment. In this embodiment, the craniomaxillofacial implant takes the form of a mandibular implant 600 to be applied to a patient's lower craniomaxillofacial bone 20, specifically to a damaged left mandible 22. Said mandibular implant 600 is formed, in accordance with the design enabled by the first aspect, into a plate that is rigid, warped, and of a substantially uniform thickness. The warpage 620 is configured preoperatively so as to adapt the mandibular implant 600 to be set onto, and supported by, at least a craniomaxillofacial bone (i.e. the left mandible 22); and support the soft tissue layer (not shown) in place of at least said craniomaxillofacial bone, thereby compensating or augmenting the elevation of the soft tissue layer. The support provided by the warpage 620 to the soft tissue layer is asymmetrical to that provided by a contralateral portion of the craniomaxillofacial bone.

The remaining detail of the mandibular implant, as well as other possible alternative embodiments for application to other species of craniomaxillofacial bone, may be appreciated by a skilled person upon the knowledge of the foregoing exemplary embodiments, and so is omitted for brevity without limitation to the concept of the present invention.

1. A method of designing a craniomaxillofacial implant that is a rigid plate of substantially uniform thickness, the method comprising positioning a surface element of the plate as a departure from a reference location within a base outline, the departure being represented by a fraction of the length of a normal path projected from the reference location to a predetermined soft tissue layer at a supported location within an anatomic region of interest, and the fraction being based upon a required patient-specific support at the supported location.

2. The method of claim 1, wherein the base outline is determined based on a postoperative craniomaxillofacial image and a preoperative craniomaxillofacial image.

3. The method of claim 2, wherein the base outline embodies a surface constraint that is determined based further on the curvature of the soft tissue layer represented by the postoperative craniomaxillofacial image.

4. The method of claim 2, wherein the required patient-specific support at the supported location is further based on a volume difference between (i) a postoperative volume of the soft tissue layer as determined from the postoperative craniomaxillofacial image and (ii) a preoperative volume of the soft tissue layer as determined from the preoperative craniomaxillofacial image.

5. The method of claim 2, wherein the postoperative craniomaxillofacial image is obtained by registering a region of anatomical reference upon the preoperative craniomaxillofacial image, the region of anatomical reference being defined correspondingly to a craniomaxillofacial region having the densest soft tissue layer that is also located closest to an operative craniomaxillofacial bone.

6. The method of claim 2, wherein the postoperative craniomaxillofacial image is generated based on a mirrored contralateral craniomaxillofacial image.

7. The method of claim 2, wherein the postoperative craniomaxillofacial image is generated based on a craniomaxillofacial template that is searchable within a library containing a plurality of the templates.

8. The method of claim 7, wherein the library is searchable by comparing anatomical landmarks of the preoperative craniomaxillofacial image with anatomical landmarks of the craniomaxillofacial templates contained within the library.

9. The method of claim 7, wherein the library is configured to update the craniomaxillofacial template based on a feedback information comprising clinical outcomes data.

10. The method of claim 9, wherein the update of the craniomaxillofacial template is carried out by an artificial intelligence.

11. The method of claim 1, wherein the fraction varies over a plurality of the reference locations within the base outline.

12. The method of claim 1, wherein the fraction is further based upon a correction factor determined by an artificial intelligence trained of clinical outcomes data.

13. The method of claim 1, wherein the length of the normal path varies over a plurality of the reference locations within the base outline.

14. The method of claim 1, wherein the required patient-specific support varies over a plurality of the reference locations within the anatomic region of interest.

15. The method of claim 1, wherein the required patient-specific support at the supported location is further based on the geometry and final position of the soft tissue layer.

16. The method of claim 1, comprising further a step of configuring porosity of the plate.

17. The method of claim 1, comprising further a step of configuring mechanical reinforcement of the plate.

18. The method of claim 1 for designing a cranial implant or a forehead implant.

19. The method of claim 1 for designing a maxilla implant or a zygomatic implant.

20. The method of claim 1 for designing an orbital implant.

21. The method of claim 1 for designing a nasal implant, a chin implant or a mandible implant.

22. A patient-specific craniomaxillofacial implant formed into a plate that is rigid, warped, and of a substantially uniform thickness, wherein the warpage is configured preoperatively so as to adapt the implant to—
be set onto and supported by at least a craniomaxillofacial bone and/or a soft tissue layer overlaying the craniomaxillofacial bone; and
support the soft tissue layer in place of at least the craniomaxillofacial bone, thereby compensating or augmenting the elevation of the soft tissue layer.

23. The implant of claim 22, wherein the warpage is configured preoperatively so as to also adapt the implant to support the soft tissue layer in place of at least the craniomaxillofacial bone, thereby compensating or augmenting the elevation of the soft tissue layer, the support being asymmetrical to that provided by a contralateral portion of the craniomaxillofacial bone.

24. The implant of claim 22, wherein the substantially uniform thickness is within a range of 0.2-0.8 mm.

25. The implant of claim 22, the soft tissue layer being one or more of muscle, fat, and skin.

26. The implant of claim 22, wherein the plate is also porous.

27. The implant of claim 26, comprising further a preoperatively configured mechanical reinforcement.

28. The implant of claim 27, wherein the mechanical reinforcement fills the porosity of the plate locally.

29. The implant of claim 27, the mechanical reinforcement thickens the plate locally.

30. The implant of claim 22, comprising further a means for fastening and/or increasing stability of the implant upon the craniomaxillofacial bone.

31. The implant of claim 22, wherein the warpage is configured preoperatively so as to adapt the implant to support the soft tissue layer by way of receiving the soft tissue layer in place of at least the craniomaxillofacial bone.

32. The implant of claim 22, wherein the warpage is configured preoperatively so as to adapt the implant to support the soft tissue layer by way of lifting the soft tissue layer over at least the craniomaxillofacial bone.

33. The implant of claim 22 for reconstructing a defective craniomaxillofacial bone and a defective soft tissue layer overlaying the defective craniomaxillofacial bone, wherein the warpage is configured preoperatively so as to adapt the implant to—
be set onto and supported by at least a non-defective part of the defective craniomaxillofacial bone and/or the defective part of the defective soft tissue layer; and
support the defective soft tissue layer in place of
the defective part of the defective craniomaxillofacial bone; and
the defective part of the defective soft tissue layer.

34. The implant of claim 33, the defective craniomaxillofacial bone being a cranium and the defective soft tissue layer comprising overlaying temporalis muscle and fat.

35. The implant of claim 33, the defective craniomaxillofacial bone being a maxilla or zygomatic and the defective soft tissue layer comprising overlaying zygomaticus major, zygomaticus minor, and fat.

36. The implant of claim 33, the defective craniomaxillofacial bone being an orbit and the defective soft tissue layer comprising overlaying orbicularis oculi, inferior rectus muscle, inferior oblique, eyeball and fat.

37. The implant of claim 22 for a cosmetic surgery, wherein the warpage is configured preoperatively so as to adapt the implant to—
be set onto and supported by at least the craniomaxillofacial bone and/or the soft tissue layer; and
support the soft tissue layer in place of at least the craniomaxillofacial bone, thereby altering the apparent contours of the craniomaxillofacial bone.

38. The implant of claim 37, the cosmetic surgery being operated upon a nasal bone or a forehead.

39. The implant of claim 37, the cosmetic surgery being operated upon a maxilla or a zygoma.

40. The implant of claim 37, the cosmetic surgery being operated upon a mandible.
